(19) United States
(12) Patent Application Publication
(10) Pub. No.: US 2020/0253986 A1
(43) Pub. Date: Aug. 13, 2020

(54) TRIPTONIDE OR A COMPOSITION COMPRISING TRIPTONIDE FOR USE IN TREATING DISORDERS

(71) Applicant: GS THERAPEUTICS LIMITED, Hong Kong (CN)

(72) Inventor: Guangbin LUO, Shaker Heights, OH (US)

(73) Assignee: GS THERAPEUTICS LIMITED, Hong Kong (CN)

(21) Appl. No.: 16/630,052

(22) PCT Filed: Jul. 10, 2018
(86) PCT No.: PCT/CN2018/095115

§ 371 Date: Jan. 10, 2020

Related U.S. Application Data
(60) Provisional application No. 62/530,845, filed on Jul. 11, 2017.

(51) Int. Cl. A61K 31/585; A61P 35/04

(52) U.S. Cl. CPC A61K 31/585; A61K 9/0019; A61P 35/04

(57) ABSTRACT

The present application provides Triptonide or a functional equivalent or pharmaceutically acceptable salt thereof, or a composition comprising the same for use in treating or preventing hyperproliferative disorders. Also provided is a method for treating or preventing hyperproliferative disorders, preferably cancer in a subject using the above substances.

### RELATED APPLICATIONS

This application claims priority to U.S. Provisional Patent Application No. 62/530,845, filed Jul. 11, 2017, the contents of which are incorporated by reference in the entirety.

### TECHNICAL FIELD

The present application relates to Triptonide or a composition comprising Triptonide, and use thereof. In particular, the present application relates to Triptonide or a composition comprising Triptonide for use or methods of treating or preventing diseases or disorders.

### BACKGROUND

Cancer is a disease of uncontrolled cell proliferation, and thus targeting cell proliferation constitutes a potentially effective strategy for combating cancer. Targeted anticancer therapy represents a revolutionary breakthrough and a new paradigm in anticancer chemotherapy. In this new paradigm, individual anticancer drugs were developed based on unique cancer-specific genotypes (mutations in specific genes) or epigenetic attributes (mis-expressions of specific genes). Accordingly, such therapies could not only facilitate the targeted killing of cancer cells to minimize the risk of severe side effects, but also enable the delivery of the treatment to those who are the most likely to benefit from the treatment, reducing the needless treatments for those who are unlikely to have a beneficial response. For this reason, targeted anticancer therapy is also referred to as personalized anticancer therapy.

However, despite the great promise of personalized anticancer therapy, to date, only a limited number of targets have been identified and successfully exploited therapeutically. Furthermore, these targeted drugs are often effective in only a small subset of patients even for those who have the same specific type of tumors with the targeted attribute. As a result, currently, personalized therapies can only benefit a very small fraction of the entire cancer patient populations. Thus, it has remained an urgent and unmet need to expand the application of this new type of therapies. Identification of new targets for developing targeted treatments represents one of the most promising avenues for achieving such an objective.

Members of the G protein-coupled receptor (GPCR) superfamily comprise the targets of many pharmaceutical drugs. These receptors transmit information from the extracellular microenvironments to the interior machinery of the cells, affecting activities of specific downstream signal transduction pathways. Protease activated receptor 2 (PAR2), encoded by the F2RL1 gene, is a member of a self-ligand GPCR sub-family of receptors for which the cognate ligand and its corresponding receptor are encoded as a single polypeptide and deployed together onto the cytoplasmic membrane.

Recent studies have revealed that PAR2 is expressed in many types of tumor cells, both under in vitro cultured conditions and in several types of human primary tumors examined, in contrast to its highly restricted expression pattern in several types of terminally differentiated non-dividing cells under the normal circumstances. Also, activation of the canonical PAR2 signaling pathway (the Gαq-PLC-IP3/DAG pathway) exhibited growth-promoting effects. Together, these observations have led to the conclusion that PAR2 activation is oncogenic. Furthermore, in some cancer cell lines, such as colon cancer-derived cell lines, suppression of PAR2 expression resulted in a growth inhibitory effect. Furthermore, in breast cancer cell lines and xenograft tumors, inhibition of the tissue factor (TF)-PAR2 pathway resulted in inhibition of cell proliferation and antitumor effect, respectively. These observations have led to the suggestion that some cancer cells are “addicted” to PAR2 activation and hence inhibiting PAR2 may have anticancer effects. However, the clinical indication of such suggestion has not been determined.

Triptonide is a natural compound that was first purified from the plant Tripterygium wilfordii in 1972 along with Tripdiolide and Triptolide (FIG. 1A). It was found that while Triptonide and Triptolide differed only at the functional group on C14, with Triptonide having a C14 ketone and Triptolide having a C14 alcohol, Triptolide but not Triptonide had a potent anti-leukemia activity. The early studies defined Triptolide as a poison with a potent anti-leukemia property. In addition, while Triptolide was reported to have modest antitumor activities in preclinical models, some diterpene lactone epoxide compounds, including Triptonide, were shown to have antifertility activities.

In the present application, the inventors studied the effect of Triptonide on cancers, especially PAR2-expressing proliferating cells. These studies provide a new anticancer paradigm of targeted therapy by the non-canonical activation of PAR2 with Triptonide and its functional equivalents.

### SUMMARY OF THE INVENTIONS

In a first aspect, the present application provides a method for treating or preventing hyperproliferative disorders, preferably cancer in a subject, which comprises administering a therapeutically or prophylactically effective amount of an agent that can cause activation of Protein Kinase A (PKA) or a pharmaceutical composition comprising the same.

In some embodiments, the treatment comprises selectively killing cancer cells, preferably PAR2-expressing proliferating cells. In some embodiments, the prevention comprises selectively killing PAR2-expressing cells in premalignant and/or malignant sites. In some embodiments, the agent that can cause activation of PKA is Triptonide, or a functional equivalent or pharmaceutically acceptable salt thereof.

In a second aspect, the present application provides a pharmaceutical composition comprising Triptonide or a functional equivalent or pharmaceutically acceptable salt thereof, and a pharmaceutically acceptable carrier.

In a third aspect, the present application provides Triptonide or a functional equivalent or pharmaceutically acceptable salt thereof, or the composition comprising the same, for use in treating or preventing hyperproliferative disorders, preferably cancer in a subject. Specifically, disclosed herein is Triptonide or a functional equivalent or pharmaceutically acceptable salt thereof, or the composition comprising the same, for use in selectively killing of cancer cells, preferably PAR2-expressing proliferating cells in a subj ect.

In a fourth aspect, the present application provides use of an agent that can cause activation of PKA in the manufacture of a medicament for treating or preventing a hyperproliferative disorder in a subject. In some embodiments, the hyperproliferative disorder is cancer.

The present application further provides use of an agent that can cause activation of PKA in the manufacture of a medicament for selectively killing of cancer cells in a subject. In certain embodiments, the cells are PAR2-expressing proliferating cells.

In some embodiments, the agent that can cause activation of PKA is an agonist for GPCR receptors. In specific embodiments, the above agent is Triptonide, or a functional equivalent or pharmaceutically acceptable salt thereof.

In a fifth aspect, the present application provides a method for treating or preventing immune response related disorders and/or pain control in a subject, comprising administering to the subject a therapeutically or prophylactically effective amount of Triptonide, or a functional equivalent or pharmaceutically acceptable salt thereof, or the pharmaceutical composition disclosed herein.

In a further aspect, the present application provides use of Triptonide, or a functional equivalent or pharmaceutically acceptable salt thereof, or the pharmaceutical composition comprising the same in the manufacture of a medicament for treating or preventing immune response related disorders and/or pain control in a subject.

In a further aspect, the present application provides Triptonide, or a functional equivalent or pharmaceutically acceptable salt thereof, or the pharmaceutical composition comprising the same for use in treating or preventing immune response related disorders and/or pain control in a subject.

In a still further aspect, disclosed herein is a method for inducing a sustained activation of PKA in PAR2-expressing proliferating cells, which comprises contacting the cells with Triptonide, or a functional equivalent or pharmaceutically acceptable salt thereof, or a pharmaceutical composition comprising the same.

In a further aspect, all the afore-mentioned compositions further comprise one or more of other agents.

In a further aspect, a method for identifying an agent that can cause the sustained activation of PKA is provided, wherein the method comprises assessing the mitotic catastrophe-inducing effect of a candidate agent. In some embodiments, the candidate agent is administered at interphase. In some embodiments, the candidate agent is administered as a short treatment of several minutes to several hours.

### BRIEF DESCRIPTION OF THE DRAWINGS

FIG. 1 shows the effect of Triptonide on cell growth. FIG. 1A illustrates the structures of Triptonide and Triptolide. FIG. 1B-1C illustrates effects of one-hour treatment with Triptonide at increasing concentrations on the growth of HepG2 (B) and cultured primary mouse hepatocytes (PMH) (C). Cells were seeded in individual wells of 96-well plates and the growth rates were assessed by the relative densities of the cultured cells while the actual images of the cultured cells were monitored by taking 4 images per well on four fixed locations every three hours using an IncuCyte Zoom system.

FIG. 2 shows the detection of cells that undergo DNA replication using the Edu-incorporation assay. HepG2 cells were synchronized by either mimosine or serum starvation. They were then released into EdU-containing medium for 30 minutes. Those that were undergoing DNA replication incorporated EdU into the newly synthesized genomic DNA. The presence of an alkyne group in the EdU-containing DNA in the proliferating cells enables the labeling and visualization of the Edu-containing DNA with a fluorescein-containing azide through “Click” reaction. The reaction between the alkyne and the azide functional groups resulted in the conjugation of the two moieties and the covalent labeling of the DNA with a fluorescent probe. Individual nuclei were visualized by staining the DNA with DAPI, a DNA specific fluorescent dye. FIG. 2A shows photographs of mimosine-treated cells after the Edu-based Click assay. The percentages of replicating cells that are positive for the Edu-based signal are presented at the bottom of the corresponding photographs. Note that majority of cells were positive for the Edu-based fluorescent signal between 0 and 1 hour after the release from the mimosine treatment. It became largely undetectable at 1.5 hours. FIG. 2B shows photographs of serum-starved cells after the Edu-based click assay. The percentages of replicating cells that are positive for the Edu-based signal are presented at the bottom of the corresponding photographs. A first wave of Edu-positive cells was observed when Edu was fed at 0 hour and began to reduce at 4 hours after the release from the starvation. A second wave of Edu-positive cells at 10 hour and diminished by 18 hours after the release.

FIG. 3 illustrates representative images of live cell imaging of serum-starved cells at different time points after release into the regular culture medium without additional treatment (Control) or with 1 μM Triptonide treatment at different time points (0, 120, and 240 minutes as TR0, TR120, TR240, respectively), demonstrating the effects of Triptonide on serum-starved HepG2 cells. The numbers on the top indicate time after the release of the cells from the serum starvation. Note that for Control and TR120, a significant increase in mitotic figures was evident at hour 13 and remained relatively constant thereafter, and that the total cell numbers have increased significantly at hour 37. In contrast, for the TR0 and TR240 treatments, total cell numbers did not change significantly throughout, but the number of cells with abnormally condensed chromatin had increased significantly by hour 37. In addition, the accumulation of the cells with abnormally condensed chromatin reached their peak values at hour 13.

FIG. 4 illustrates representative images of mimosine treated HepG2 cells treated with either the vehicle only (Control) or 1 μM Triptonide at different time points (0, 60, 120, and 240 minutes, or, TR0, TR60, TR120, TR240, respectively, after the mimosine treatment) and then returned back to the regular culture medium, demonstrating the effects of Triptonide on mimosine-treated HepG2 cells. Images taken at 0, 14, 23, and 37 hours after the initiation of the experiments are shown. The numbers on the top indicate time (hours) after the cells were released from the mimosine treatment. Note the increases in total cell numbers from the left to the right for the control (the top row), the TR60 and TR240 treatments; the lack of such a change in the TR0 and TR120 samples; and the massive accumulation of cells with abnormally condensed chromatin for the TR120 treatment (the far right image of TR120). Note also the appearance of a relatively constant number of the smaller dark and round entities of metaphase cells starting at hour 14 for the Control, TR60, and TR240; and the steady increase in number of cells with abnormally condensed chromatin for the TR120 treatment starting at hour 14.

FIG. 5 illustrates images of the mimosine-treated cells that were further treated with the vehicle solution (Control), 1 μM, or 2 μM of Triptolide for a one-hour at 0 or 120 minutes after the initiation of the experiments, demonstrating the effects of Triptolide on mimosine-treated HepG2 cells. Only images taken at 0 and 37 hours after the initiation of the experiments are shown. Note the dramatic accumulations of cells with condensed chromatin at 37 hours after the initiation only for those treated with 2 μM Triptolide at both 0 and 120 minutes (TR0-2 TR120-2 μM). Those that were treated with 1 μM Triptolide (TR0-1 TR120-1 μM) did not exhibit such a feature.

FIG. 6 shows the effect of Triptonide and Triptolide on cell cycle progression of HepG2 cells. Asynchronous (AS) HepG2 cells were treated with 200 μM mimosine for 28 hours and then return to regular culture medium alone for 0, 11, 24, and 37 hours (Mim 0, Mim R11, Mim R24, Mim R37) (Top panel), or with 1 μM Triptonide, 2 μM Triptolide, or 10 μM Triptonide (bottom panel), respectively. A DNA content-based flow cytometry was then performed to assess the composition of cells at various stages of the cell cycle, i.e. G1: 2N; S: >2N to <4N; G2/M: 4N; sub-G1: <2N, respectively. The percentages of the G2/M phase (4N) cells are shown. Note the sustained high percentages of the G2/M subpopulation in the cells treated with 1 μM Triptonide and the significant peaks of the sub-G1 (<2N, indicative of apoptosis) population in the cells treated with either 2 μM Triptolide or 10 μM Triptonide, but not in those treated with 1 μM Triptonide.

FIG. 7 shows the effects of Triptonide on cultured primary keratinocytes. FIG. 7A-7C illustrates growth curves of wild-type keratinocytes (A), or Par2 knockout keratinocytes (B, C) after being treated for one hour with various concentrations of Triptonide. The corresponding IC50s were included. FIG. 7D-7E illustrates growth curves of wild-type keratinocytes (D), or Par2 knockout keratinocytes (E) after being exposed continuously to various concentrations of Triptonide. FIG. 7F shows growth curves for wild-type keratinocytes after the treatment with various concentration of trypsin for 30 minutes. Note: 1). The unique hypersensitivity of wild-type but not Par2 knockout keratinocytes to the one hour treatments of Triptonide at concentrations below 5 μM (A, and B); 2). The growth inhibitory effects of the Par2 knockout cells of Triptonide at 100 and 200 μM; 3). The lack of a significant effect from the Trypsin treatment (F).

FIG. 8 illustrates images of Western blots showing the presence or absence of the PAR2- or Par2-specific bands as well as those β-actin (ACTB, as a loading control) in primary mouse hepatocytes (PMH), immortalized human hepatocyte cell line LO2, and hepatocellular cancer cell line Hep3B and HepG2, which demonstrate PAR2 expression in immortalized human hepatocyte cell line LO2 and human liver cancer cell lines, but not in primary mouse hepatocytes.

FIG. 9 shows the effects of Triptolide on cultured primary keratinocytes. FIG. 9A-9B illustrate growth curves of wild-type keratinocytes (A), or Par2 knockout keratinocytes (B) following the exposures to Triptonide at various concentrations for one hour. FIG. 9C-9D illustrate growth curves of wild-type keratinocytes (C), or Par2 knockout keratinocytes (D) following the continuous exposure to Triptolide at various concentrations. Note the lack of any significant growth inhibitory effects of the one hour treatments of Triptolide at concentrations up to 800 nM and the similar potent growth inhibitory effects of Triptolide at as low as 1.25 nM when applied in a continuos fashion.

FIG. 10 shows the effects of Trypsin, Triptonide and Triptolide on ERK phosphorylation in HepG2 cells. HepG2 cells were serum-starved for 48 hours and then cultured in serum free basal medium 1640 with the DMSO vehicle, trypsin (50 nM), Triptolide (1 μM), or Triptonide (1 μM). Samples were taken for total protein extraction at various time points (0, 5, 10, 20, and 40 minutes, respectively). Western blots were performed with antibodies specific for phosphorylated ERK (p-ERK), unphosphorylated ERK (ERK), and β-actin, respectively. Note the diffences in ERK phosphorylation following the treatments with trypsin (50 nM), Triptolide (1 μM), or Triptonide (1 μM), respectively.

FIG. 11 shows the effects of Triptonide on the levels of phosphorylated histone H3 in HepG2 cells. HepG2 cells were synchronized by mimosine for 28 hours and released into regular culture medium for 2 hours. The cells were then treated with the vehicle solution or 1 μM Triptonide for 1 hour. After the treatment, cells were incubated in the regular culture condition and samples were harvested every hour from 2 to 12 hours (shown on top of the top panel). Western blots were performed to determine the relative levels of phosphorylated histone H3 (p-H3), CDK1 and beta actin (ATCB) (as controls). Note that the peak level of p-H3 was detected in the control sample harvested at 10 hours after the treatment with the vehicle solution, and the lack of significant increases in levels of p-H3 in the samples derived from the cells that were treated with Triptonide.

FIG. 12 shows the effects of Trypsin and Triptonide on the levels of cAMP in HepG2 cells. HepG2 cells were synchronized by mimosine for 28 hours then released into regular culture medium for two hours (Triptonide-2, when the cells were sensitive to Triptonide's mitotic catastrophe-inducing effect). The cells were then treated with the vehicle solution, 50 nM Trypsin, or 1 μM Triptonide in serum free basal medium for various times before samples were harvested. In addition, a set of cells was allowed to recover for 4 hours in regular medium (Triptonide-4, when the cells were not sensitive to Triptonide's mitotic catastrophe-inducing effect) before treating with 1 μM Triptonide. The levels of cAMP in each sample were determined. Note the modest and transient elevation in the levels of cAMP in the Trypsin-treated cells and the much higher levels of two recurrent peaks of cAMP in cells treated with Triptonide at 2 (Triptonide-2), but not 4 (Triptonide-4) hours after the cells were allowed to recover in regular culture medium.

FIG. 13 shows the effects of Triptonide on the levels of PKA activities in HepG2 cells. HepG2 cells were synchronized by mimosine for 28 hours then released into regular culture medium for two hours. The cells were then treated with the vehicle solution, or 1 μM Triptonide in serum free basal medium for various times before samples were harvested. The levels of PKA activities in each sample were then determined. Note that the two peaks of elevation in PKA activities are much higher in the Triptonide-treated cells than those in the untreated cells.

FIG. 14 shows the effects of Trypsin and Triptonide on the levels of PKA activities in HepG2 cells. HepG2 cells were synchronized by mimosine for 28 hours then released into regular culture medium for two hours. The cells were then treated with the vehicle solution, 50 nM Trypsin, or 1 μM Triptonide in regular culture medium for one hour before reruning to regular culture medium. Samples were harvested at various times after each treatment, and the activity of PKA for each sample was determined. Note that for the untreated cells or those treated with Trypsin, dramatic reductions in the levels of PKA activities occurred at 9 hours after the release from the mimosine treatment; while for those treated with Triptonide, the levels of PKA activities were not reduced significantly at the same time point.

FIG. 15 shows the impact of blocking the AC-cAMP-PKA signaling pathway on the effect of Triptonide on HepG2 cells. HepG2 cells were seeded in 96-well overnight and then treated with the vehicle solution (Control, Ctrl), Triptonide (Trip, 1 μM), Vidarabine (Vid, 10 μM), myristoylated PKI-14-22 amide (PKI, 2.5 μM), Triptonide plus Vidarabine (Trip+Vid), or Triptonide plus PKI (Trip+PKI). Cell growth and morphology were monitored by using the IncuteCyte Zoom system. FIG. 15A illustrates the effect of Vidarabine, Triptonide, and Vidarabine plus Triptonide. Note that Vidarabine has no significant effects on the growth of the cells. Triptonide is growth inhibitory. Vadarabine plus Triptonide does not have a significant effect on cell growth either. FIG. 15B illustrates the effect of PKI, Triptonide, and PKI plus Triptonide. Note that PKI alone has no significant effects on the growth of the cells. Triptonide is growth inhibitory. Triptonide plus PKI does not have a significant effect on cell growth.

FIG. 16 illustrates images of western blot analysis showing the presence or absence of the PAR2 as well as those β-actin (ACTB, as a loading control) in immortalized gastric epithelial cell line GES-1 and 5 gastric cancer cell lines, which demonstrate PAR2 expression in gastric cancer cell line and in the immortalized gastric epithelial cell line.

FIG. 17 shows the effects of Triptonide on tumor-bearing mice. FIG. 17A illustrates photographs of GFP-fluorescent live imaging of three tumor-bearing mice representing the three different treatment cohorts (n=10 for each cohort) at different time points (days) after the initiation of the treatments with the vehicle for the Triptonide treatment (the top row), Triptonide at the 25 mg/kg body weight level (by gavage), or Sorafenib (by introperitoneal injection). Note that both the intensities and the relative areas of the GFP signals increase progressively over time for the vehicle-treated mouse (top row), while those for the Triptonide-treated one decline starting at Day 4 after the treatment and became undetectable by Day 11. Those for the Sorafenib-treated group became stable initially. They then exhibited a brief reduction, but then began to increase. FIG. 17B illustrates growth curves of tumor cells of the three different cohorts reflected by the average areas of individual tumors of each cohort. The GFP-fluorescent signal was no longer detected in any of the 10 tumor-bearing mice in the Triptonide treated group by Day 18 and beyond. FIG. 17C illustrates body weight curves for each of the three treatment cohorts. Note the lack of any significant differences among the three group.

### DETAILED DESCRIPTION OF INVENTIONS

The present application provides an agent that can cause activation of protein kinase A, or a pharmaceutical composition comprising the same for use in treating or preventing hyperproliferative disorders (especially cancer), immune response related disorders and/or pain control in a subject, or for use in a method for treating or preventing these disorders or diseases.

In certain embodiments, the agent that can cause activation of protein kinase A disclosed herein is an agonist for GPCR receptors. In preferable embodiments, the agent is Triptonide, or a functional equivalent or pharmaceutically acceptable salt thereof.

In certain embodiments, the agent that can cause activation of protein kinase A can selectively killing cancer cells in a subject. In specific embodiments, the cells are PAR2-expressing proliferating cells.

In some embodiments, the cancer can be a primary cancer or a metastatic cancer. In specific embodiments, the cancer can be hepatocellular carcinoma, breast cancer, colon cancer, non-small cell lung cancer, gastric cancer, ovarian cancer, renal cancer, prostate cancer, central nerve system cancer, melanoma and the like.

The availability of the desirable novel targets is a limiting factor for developing new targeted anticancer therapies. This disclosure describes the identification of PAR2 as a novel target that can be exploited for instigating selective killing of PAR2-expressing cancer cells through a unique mode of PAR2 activation by using Triptonide or its functional equivalents. Significantly, our studies found that Triptonide could be used to cause mitotic catastrophe by the non-canonical activation of PAR2 that is associated with a prolonged elevation of PKA, enabling the targeted killing of PAR2-expressing, proliferating cells. Unexpectedly, our data have revealed that although PAR2 expression is primarily restricted to quiescent and/or terminally differentiated non-dividing cells, it is expressed in two transformed human cell lines (LO-2 and GES-1) as well as many human cancer cell lines. This finding provided evidence that abnormal activation of PAR2 could represent an early “driver” alteration (i.e. a driver change) that has occurred before or during oncogenesis and hence a desirable target for instigating the specific killing of cancer cells for therapeutic benefits without causing unacceptable harmful side effects. Importantly, we have shown that orthotopical HepG2 xenograft tumors could be rapidly and completely eliminated from the tumor-bearing mice by a treatment with Triptonide that is many folds below the maximum tolerable dose, providing the proof-of-principle for this new paradigm of targeted anticancer therapy. In some embodiments, given the wide-spread expression of PAR2 in human cancers and the early onset of PAR2 activation during oncogenesis, such a paradigm could be applicable for the treatment and/or prevention of many types of human cancers. In some embodiments, given that PAR2 plays a significant role in both inflammatory response and pain control and the great safety profile of Triptonide, it is possible that Triptonide could be exploited for the management of disorders associated with inflammation and excessive pain by modulating the PAR2-mediated inflammatory and/or pain responses.

Cancers is a disease of abnormal cell proliferation and therefore killing and/or suppressing the growth of the abnormally proliferating cancer cells constitute a main strategy for treating this disease. Proliferation of human cells, both normal and malignant, is a highly regulated and complex process. In humans, a given cell, once born, could either stay in a quiescent (also known as G0) state, or go on to a new round of proliferation, giving rise to two new daughter cells. A cell proliferation cycle is divided into four sequential phases: the gap 1 (G1), the synthesis (S), the gap 2 (G2) and the mitosis (M) phase. More recently, the G1 phase has been further subdivide into an early G1, or G1-postmitosis (G1-ps) and a late G1 or G1-pre-S (G1-ps). The G1-pm defines a relatively constant period (3-4 hours) that represents the minimum time for the newly born cells to become competent to pass the so-called restriction (or R) point to commit to proliferation; while the G1-ps is much more variable among different cell types or even among individual cells of the same type. Before passing the “R” point, individual cells retain the capacity of exiting the cell cycle to enter a quiescent or G0 state. Activation of the MAPK-ERK pathway by mitogenic stimulations constitutes an important force that drives individual cells to pass the “R” point and to commit to proliferation. Once committed into the cell cycle, the progression of the cell cycle is subjected to additional regulations including those at the G1/S, S/G2, G2/M boundaries and during mitosis. Once a cell passes the G2/M transition, it will be either arrested by the antephase checkpoint or progress into prometaphase. Cells that are arrested at the antephase checkpoint can withdraw back into an interphase state and then could resume forward progression once the conditions become appropriate. In contrast, those that have entered the prometaphase have passed the so-called “point-of-no-return” and can no longer withdraw back to an interphase state. Rather, they could either progress normally to complete a productive mitosis with the production of two diploid daughter cells; or end with a failed mitosis. Some failed mitoses could lead to the formation of tetraploid cells, while the rest will ultimately succumb to death, i.e. result in mitotic catastrophe. Thus, in order to prevent failed mitoses, G2/M transition is highly regulated.

In mammalian cells, mitosis promoting factor (MPF) plays a critical role in regulating G2/M transition. The core component of this MPF is the cyclin B1-cyclin dependent kinase 1 (CDK1) kinase complex. The activation of the CDK1 kinase is both necessary and sufficient to facilitate the G2/M transition to initiate mitosis. During the interphase of the cell cycle, this kinase complex is inactive as the result of phosphorylation by the Wee1/Tyt1 kinases. At late G2, CDK1 is activated by the action of CDC25 phosphatase, which removes the inhibitory CDK1 phosphorylation. More recent studies have shown that G2/M transition is initiated first by activating the cyclin B1-CDK1 complex in the cytoplasmic compartment. This is then immediately followed by the import of the activated cyclin B1-CDK1 complex into the nucleus, leading to the highly coordinated events that are associated with mitosis. Intriguingly, in the mammalian female oocytes, the G2/M transition at the prophase I of meiosis is suppressed by the inactivation of initial activation of CDK1 in the cytoplasm through the elevation of cytoplasmic PKA activity. The elevation of the cytoplasmic PKA activity suppresses CDK1 activation by phosphorylating a number of proteins, including Weel and CDC25, which results in Wee1 activation and CDC25 inactivation, respectively. Since Weel activation and CDC25 inactivation both suppress CDK1 activation, this elevation of PKA activity provides a very effective mechanism for suppressing G2/M transition. Elevation of PKA activity is also an effective means for suppressing G2/M transition in mammalian somatic cells.

PAR2 is a member of a self-ligand GPCR sub-family of receptors for which the cognate ligand and its corresponding receptor is encoded as a single polypeptide and deployed together onto the cytoplasmic membrane. The canonical ligand of PAR2 is located in the N-terminal of the polypeptide and is positioned outside the cytoplasmic membrane in an unavailable state. It becomes available when the protein is cleaved by a specific protease, such as Trypsin. The two major biological roles of PAR2/Par2 are: 1) A sensory role in pain and itch perception in the nervous system; 2) A role in the regulation of barrier integrity and the inflammatory response in the epithelial linings of the various organs/tissues. Accordingly, human PAR2 and its mouse homologue Par2 are expressed primarily at high levels in the terminally differentiated epithelial cells of the epidermis, on the top of the crypts of the gastrointestinal tract, and in a subset of neurons. Par2 deficient mice are viable and developmentally normal, but have defects in pain perception and in inflammatory response. Thus, the pattern of PAR2/Par2 expression correlates remarkably well with its defined roles in vivo. The important roles of PAR2 in immune response and pain control have led to a great interest in the development of PAR2 modulators as potential drug candidates for managing conditions associated with pain and/or inflammation. Such modulators have proven very useful both as research tools as well as potential leads for developing drugs for treatments related to immune response and/or pain control. Of note, these modulators include small molecule modulators that target the canonical PAR2-mediated signaling pathway, i.e. the Gaq-PLC pathway.

Alternatively, PAR2 activation can be induced by some proteases such as elastin and cathepsin, which can lead to the activation of the Gαs-cAMP-PKA signaling cascade, resulting in the activation of the PKA kinase. The Inventors first discovers Triptonide as the very first small modulator for the PAR2-mediated Gαs-AC-cAMP-PKA pathway.

In some embodiments, the agent disclosed herein such as Triptonide, or a functional equivalent or pharmaceutically acceptable salt thereof can activate PAR2, and in turn cause sustained activation of PKA. In preferable embodiments, the agent disclosed herein such as Triptonide, or a functional equivalent or pharmaceutically acceptable salt thereof produces a mitotic catastrophe-inducing effect, thereby leading to the ultimate demise of proliferating cancer cells.

In some embodiments, the agent disclosed herein such as Triptonide, or a functional equivalent or pharmaceutically acceptable salt thereof can be used to facilitate the selective killing PAR2-expresing proliferating cells without causing unacceptable adverse effects.

The inventors have demonstrated that Triptonide had the desirable selective lethal effect on proliferating cells including cancer cells, which is in part due to Triptonide's unique mitotic catastrophe-inducing effect on mitogenically activated cells while sparing the quiescent, non-dividing cells.

In specific embodiments, Triptonide is identified as a unique agent that can be used to induce PAR2/Par2-mediated mitotic catastrophe in Par2/PAR2-expressing cells, leading to the selective killing of proliferating Par2/PAR2-expressing cells. The inventors found that the mitotic catastrophe-inducing effect of Triptonide is due to its non-canonical agonist effect on the Par2/PAR2-Gαs-AC-cAMP-PKA signaling cascade.

In some embodiments, Triptonide functions as an unusual PAR2 agonist, leading to abnormal activation of the AC-cAMP-PKA pathway. The inventors first identify Triptonide as a small molecule agonist for the PAR2-Gαs-AC-cAMP-PKA signaling pathway.

In some embodiments, the duration of exposure of cancer cells to Triptonide or a functional equivalent or pharmaceutically acceptable salt thereof allows sustained activation of PKA kinase and in turn cancer cell-specific growth inhibition without causing PAR2/Par2-independent harmful effects.

In specific embodiments, the cancer cells are exposed to Triptonide for multiple times. It is preferable that a time span between successive exposures that is sufficient for the significant clearance of the administered Triptonide is implemented so that a possible PAR2/Par2-independent harmful effect would not occur or would not reach an unacceptable level.

In specific embodiments, the duration of exposure or the time span between successive exposures can be determined by a skilled person in the art as needed in view of the disclosure herein. For example, the desired duration of exposure for HepG2 cells can be 20 minutes to 2 hours, e.g. one hour. For another example, the time span between two successive dosages in tumor-bearing mice by gavage can be one day, two days, three days, etc.

In one aspect, a pharmaceutical composition comprising Triptonide or a functional equivalent or pharmaceutically acceptable salt thereof is provided. In some embodiments, the pharmaceutical composition can further comprise one or more of other agents through the so-called combinatorial strategy to enhance the desired therapeutic efficacy, to reduce the undesirable effects, or both.

The pharmaceutical composition disclosed herein can be presented in the form of a unit dose, and can be prepared by any methods well known in the pharmaceutical field. All of the methods comprise the step of combining the active ingredients disclosed herein with one or more pharmaceutically acceptable carriers or other agents or other forms of interventions. Generally, a composition is prepared by combining active ingredients with a liquid carrier or a solid carrier or both, followed by shaping the resultant product as required.

In certain embodiments, Triptonide, or a functional equivalent or pharmaceutically acceptable salt thereof disclosed herein, or the composition comprising the same can be formulated together with a pharmaceutically acceptable carrier into pharmaceutically acceptable dosage forms, for example, oral liquid, capsule, powder, tablet, granule, pill, syrup, injection, suppository and the like, either by itself, or in combination with other agents or other forms of interventions.

The “pharmaceutically acceptable carrier” disclosed herein refers to a carrier which does not interfere with the bioactivity of active ingredients, including those commonly used in the pharmaceutical field. The pharmaceutically acceptable carrier disclosed herein can be solid or liquid, including pharmaceutically acceptable excipients, buffers, emulsifiers, stabilizers, preservatives, diluents, encapsulants, fillers and the like. For example, the pharmaceutically acceptable buffer further comprises phosphates, acetates, citrates, borates, carbonates and the like.

In certain embodiments, Triptonide or a functional equivalent or pharmaceutically acceptable salt thereof, or the composition comprising the same is administered via any appropriate routes, such as orally, subcutaneously, intramuscularly or intraperitoneally. In preferred embodiments, Triptonide or a functional equivalent or pharmaceutically acceptable salt thereof, or the composition comprising the same is administered orally, either by itself, or in combination with other agents or other forms of interventions.

In another aspect, a method for treating or preventing hyperproliferative disorders such as cancer, immune response related disorders and/or pain control in a subject is provided, which comprises administering a therapeutically or prophylactically effective amount of Triptonide, or a functional equivalent or pharmaceutically acceptable salt thereof, or a pharmaceutical composition comprising the same to the subject, either by itself, or in combination with other agents or other forms of interventions.

In a further aspect, use of a therapeutically or prophylactically effective amount of Triptonide, or a functional equivalent or pharmaceutically acceptable salt thereof, or a pharmaceutical composition comprising the same in treating or preventing hyperproliferative disorders such as cancer, immune response related disorders and/or pain control in a subject is provided, either by itself, or in combination with other agents or other forms of interventions.

In certain embodiments, the treatment comprises selectively killing cancer cells, preferably PAR2-expressing proliferating cells. In some embodiments, the prevention comprises selectively killing PAR2-expressing cells in premalignant and/or malignant sites.

The disclosure described herein provides an effective targeted anticancer therapy. The recent advent of targeted anticancer therapies has provided great hope for cancer patients. Traditionally, the development of a targeted anticancer therapy begins with the identification of a cancer specific attribute (for example, a cancer specific mutation or gene expression signatures), followed by the development of the appropriate modulating agents. However, such a strategy has been proven less fruitful for developing targeted therapies for hepatocellular carcinomas (HCCs), and thus the development of effective targeted anticancer drugs had remained an unmet urgent need.

In some embodiments, the disclosure described herein provides an effective targeted therapy for hepatocellular carcinomas. In other embodiments, the disclosure described herein provides effective targeted therapies for breast cancer, colon cancer, non-small cell lung cancer, gastric cancer, ovarian cancer, renal cancer, prostate cancer, central nerve system cancer, melanoma, etc.

The “therapeutically effective amount” or “prophylactically effective amount” used herein can be determined as the case may be, and can be easily operated by a person having ordinary skills in the art according to the dosage actually required, for example, according to the body weight, age and conditions of a patient, and/or with the available arts in the field of personalized medicine. Where the composition comprises a pharmaceutically acceptable carrier, the active ingredients and the carrier can be mixed by conventional methods in the pharmaceutical field to manufacture the required medicament.

In certain embodiments, when Triptonide, or a functional equivalent or pharmaceutically acceptable salt thereof is administered in combination with one or more of other agents or form(s) of interventions in order to enhance the beneficial effect, to reduce the undesirable effects, or both.

The term “subject” used herein refers to a mammal, including but not limited to primates, bovine, horses, pigs, sheep, goats, dogs, cats, and rodents such as rats and mice. The cells used herein can be from a subject, an organ, a tissue, a cell or any other suitable sources.

In the instant specification and claims, the terms “comprise/comprises/comprising”, “include/includes/including” and “contain/contains/containing” mean “including but not limited to”, and are not intended to exclude other parts, additives, components or steps.

It should be understood that the features, characteristics, components or steps described in the specific aspects, embodiments or examples in the present application can be applied to any other aspects, embodiments or examples described herein, unless indicated to the contrary.

The above disclosures describe the inventions in general, and the following examples further illustrate the inventions. These embodiments, examples and figures described are only intended to illustrate the inventions, but not considered as any limitation to the inventions. The sole and exclusive indicator of the scope of the invention, and what is intended by the applicant to be the scope of the invention, is the literal and equivalent scope of the set of claims that issue from this application, in the specific form in which such claims are issued, including any subsequent correction or corrections. Although specific terms and values are used herein, these terms and values should also be interpreted to be illustrative, and do not limit the scope of the inventions. Unless indicated specifically, the experimental methods and techniques in the specification are methods and techniques well known to a person skilled in the art.

### EXAMPLES

### Experimental Methods

### 1. Cell Culture Experiments

### 1.1 Preparation and Culture of Primary Mouse Hepatocytes and Keratinocytes

The initiation and culture of primary mouse hepatocytes and keratinocytes were carried out as previously described. Briefly, to initiate hepatocyte culture, animals were first anesthetized with pentobarbital sodium (400 mg/kg, ip), then the peritoneal cavity was opened, and the liver was perfused in situ via the portal vein for 4 min at 37° C. with calcium-free HEPES buffer and for 8 to 10 minutes with HEPES buffer containing 0.5 mg/ml collagenase D (Life Technologies, USA) and 3 mM CaCl₂. The perfusion rate was set at 5 ml/min. The cells were seeded onto individual well of 12-well plates (Corning, USA) at a density of 400,000 cells/well in Williams' medium E (Life Technologies, USA) supplemented with 10% fetal bovine serum (Life Technologies, USA) and allowed 2 hours to attach. Unattached cells were discarded, while the attached cells (the hepatocytes) were kept in fresh culture medium.

For the neonatal mouse keratinocyte culture, the dorsal skins of neonatal wild type or Par2 knockout mice were harvested from wild type or Par2 knockout mice, respectively. The skins were incubated overnight at 4° C. in a 0.25% solution of trypsin (Life Technologies, USA) in phosphate-buffered saline (PBS) without calcium and magnesium. The epidermis was then separated from the adjoining dermis, and the dispersed epidermal cells were collected in suspension Eagle's minimal essential medium (SMEM) (Life Technologies, USA) supplemented with glutamine and 8% calcium free fetal calf serum (FCS) (Life Technologies, USA). The cells were plated onto individual wells of 48 well plates (Corning, USA), which were pre-coated with collagen (Life Technologies, USA), at a density of 70,000 cells/well. The cell cultures were kept for 12 hours at 34° C. in a humidified incubator containing 8% CO₂. Low calcium (0.05 mM) S-MEM, containing 8% FCS was then added to initiate the culture. The culture medium was changed every 2 days.

### 1.2 Culture of Established Cell lines

Cancer cell lines and immortalized human cell lines were cultured in 1640 tissue culture medium (Corning, USA) with 10% fetal bovine serum in a standard tissue culture condition of 37° C. and 5% CO₂.

### 1.3 HepG2 Cell Synchronization

HepG2 cells that are approximately 60% confluent were maintained in a serum-free 1640 medium (Corning, USA) for 48 hours in the absence or presence of 200 mimosine for 28 hours.

### 2. IncuCyte Zoom-Based Proliferation Experiments

For primary hepatocytes, the seeded cells were monitored for up to 48 hours either without any treatment (as a control) or with various types of treatments. For primary keratinocytes, cells in 48-well plates were monitored either without any treatment or with various types of treatment for up to 96 hours. For HepG2, cells 96-well plates were monitored either without any treatment or with various types of treatments for up to 48 hours. The IncuCyte Zoom was set to take a set of images at fixed locations (4 per well for 96-well plates and 16 per well for 48-well plates and 12-well plates) every three hours.

### 3. Western Blot

After treatments, cells were harvested and protein extracts were prepared by lysis in RIPA buffer (Solarbio). Western blots were performed using specific antibodies and SuperSignal® West Pico Chemiluminescent Substrate (Pierce). Antibodies were purchased from commercial venders as described below: polyclonal rabbit anti-PAR2 antibodies (ABGENT), polyclonal rabbit anti-Beta-Actin antibodies (Cell Signaling Technology), anti-ERK (Cell Signaling Technology), anti-phosphorylated ERK (Cell Signaling Technology), anti-phosphorylated histone H3 (Abchem), anti-CDK1 (Abcam).

To assess the effect of Triptonide on ERK phosphorylation, HepG2 cells were seeded in 6 well plates at 2×10⁵ cells per well, cultured for 12 hours and then synchronized by serum starvation in serum-free 1640 basal medium for 48 hours. The cells were then immediately treated with either the vehicle solution or with 1 μM Triptonide. Samples were taken at various time points after the treatments for Western blot analysis.

To assess the effect of Triptonide on histone H3 phosphorylation, HepG2 cells were seeded in 6 well plates at 2×10⁵ cells per well, cultured for 12 hours and then synchronized in the culture medium containing 200 μM mimosine for 28 hours. Following the mimosine treatment, cells were recovered in regular culture medium for 2 hours and then treated with either the vehicle solution or 1 μM Triptonide for one hour. After the Triptonide treatments, cells were returned to regular culture condition and samples were harvested at various time points for Western blot analysis.

To assess the levels of PAR2 expression in cultured cells, various cell lines or primary cells were cultured to approximately 90% confluent before being harvested for Western blot analysis.

### 4. Edu-Incorporation and Detection Experiments

The experiments were carried out using the “Click-iT Plus EdU Imaging Kits” (Life Technologies, Carlsbad, Calif., USA) according to the instruction provided by the manufacturer with minor modifications. Briefly, HepG2 cells were seeded onto coverslip inside individual wells of 24-well plates (one coverslip per well) at a density of 30,000 cells/well. The HepG2 cells were either untreated, or at various duration of recovery in regular medium after subjecting to either 28 hours of treatment with 200 μM, or incubation in serum free medium for 48 hours. The cells were then released back into a drug-free medium for various times before being fed with Edu (at 10 μM) for 30 minutes. The cells were then fixed with 3.7% formaldehyde in PBS for 15 minutes at room temperature, followed by permeabilization using 0.5% Triton X-100 for 20 minutes at room temperature. Click-iT Plus reaction cocktail was then added and incubated for 30 minutes. The coverslips were washed with 3% BSA in PBS, and then stained with Hoechst 33342 (5 μg/ml). The Click reaction is designed to attach the Alexa Fluor florescent dye onto Edu, enabling the visualization of Edu-containing DNA and the cells that were undergoing DNA synthesis when Edu was added. The Hoechst 33342 fluorescent dye binds specifically to DNA, allowing the visualization of all nuclei. Following the Click reaction and Hoechst 33342 staining, fluorescent microscopy was performed to assess the Edu-incorporating characteristics of cells that had been subjected to various types of treatments.

### 5. Preclinical Antitumor Experiments

The preclinical experiments were carried out by AntiCancer Biotech (Beijing) Co., Ltd.

### 5.1 Animals

Female athymic nude mice (6 weeks of age) were used in this study. The animals were purchased from Beijing HFK Bioscience, Co., Ltd and maintained in a High Efficiency Particulate Air Filter (HEPA) filtered environment with cages, food, and bedding sterilized by irradiation or autoclaving. A total of 30 nude mice were used for the study.

### 5.2 Reagents Used for the Study

Triptonide-containing suspensions were prepared as ready-to-administer oral formulations in carboxy methyl cellulose suspension. The concentrations of Triptonide were selected so that the desired amount of the drug for each animal could be obtained in a volume of approximately 0.2 ml. During the course of the animal study, the vehicle suspension and the Triptonide-containing suspension were designated as Reagent A and Reagent B. The information regarding the nature of Reagent A and Reagent B were withheld from AntiCancer Biotech (Beijing) Co., Ltd., which performed the animal experiments.

### 5.3 Tumor Cells

HepG2-GFP human hepatocellular carcinoma cells (AntiCancer, Inc., San Diego, Calif.) were incubated with RPMI-1640 (Gibco-BRL, Life Technologies, Inc.) containing 10% FBS. Cells were grown in a CO₂ Water Jacketed Incubator (Forma Scientific) maintained at 37° C. and a 5% CO₂/95% air atmosphere. Cell viability was determined by trypan blue exclusion analysis.

### 5.4 Subcutaneous Human Hepatocellular Carcinoma Model

Female athymic nude mice were each injected subcutaneously with a single dose of 5×10⁶ HepG2-GFP cells. Tumors were harvested when their sizes reached approximately 1 cm³.

### 5.5 Orthotopic Human Hepatocellular Carcinoma Model

The method for establishing the Orthotopic Human Hepatocellular Carcinoma Model has been described previously (62). HepG2-GFP cells derived subcutaneous tumors were divided into fragments of approximately 1 mm³ and implanted orthotopically into the right lobe of the liver of 6-week old female BALB/cnu nude mice (Beijing HFK BioScience Co., Ltd.), one implant per animal. Briefly, an upper abdominal incision of 1 cm was made under anesthesia. Right lobe of the liver was exposed and a part of the liver surface was injured mechanically by scissors. Then a piece of tumor fragment was fixed within the liver tissue, and the liver was returned to the peritoneal cavity followed by the abdominal wall closing. Mice were kept in laminar-flow cabinets under specific pathogen-free-conditions.

### 5.6 Study Design

At three days after the implantation, animals with an implanted tumor of approximately 2 mm² were selected based on the results of fluorescent imaging. For this experiment, animals with the desired tumors were divided randomly into groups, 10 animals per group. Also, individual mice were each given an earmark for identification.

### 5.7 Treatments

First, a preliminary experiment was carried out to determine the maximal non-toxic dose by administering an oral dose of Triptonide at 0, 5, 10, 25, 50, 100, 200 mg/kg body weight once every other day. This experiment revealed that dose levels at up to 100 mg/kg body weight did not cause any significant adverse effects. Thus, a second preliminary experiment was carried out in tumor-bearing nude mice to determine whether an antitumor effect could be achieved within the non-toxic range. In the first set of the experiments, tumor-bearing mice were treated with 0, 1, 5, 10, 25 mg/kg Triptonide by gavage with the regimen of one dosage every other day. The results showed that the treatment with the 25 mg/kg dosing was very effective in reducing the tumor mass within a week. Thus, the 25 mg/kg once every other day regimen was selected for the preclinical experiment. The volumes of individual tumors were assessed by bioluminescent imaging.

### 5.8 Animal Monitoring

During the period of the study, all of the experimental mice were checked daily for mortality or signs of distress. The animals were observed until day 28 after tumor implantation.

a. Body Weights

The body weights of the mice were measured every three days during the study period.

b. Whole-Body Imaging

Images of tumor growth and progression were acquired every three days during the period of the study with the FluorVivo imaging system, Model 300/Mag (INDEC, Calif., USA).

c. Termination

Animals were euthanized by injection of over-dose pentobarbital sodium.

d. Autopsy

As soon as each animal was euthanized, its liver was exposed by removing the skin and immediately subjected to a final GFP fluorescent imaging. After imaging, the site for the implantation of each liver was examined for tumor or its remnant. When a tumor was clearly identifiable, it was excised and its weight was determined using an electronic balance (Sartorius BS 124 S, Germany). Tumor tissues or tissues around the implanted sites when tumors were not visible were harvested and kept in formalin for further analysis.

### 6. Cell Cycle Analysis and Flow Cytometry

A standard propidium iodide (PI) staining approach was used to analyze the cell cycle profile. Briefly, HepG2 cells were seeded and then treated either with or without 200 μM mimosine for 28 hours. The untreated cells were used as an asynchronized control. Some of the mimosine treated cells were then cultured in the absence of additional drug treatments and were harvested at 0, 11, 24 and 37 hours for flow cytometry analysis. Alternatively, the same mimosine treated cells were cultured in the presence of 1 μM Triptonide, 2 μM Triptolide, or 10 μM Triptonide and were harvested at the same times points as those for the untreated cells. The cells were fixed with 70% ethanol in −20° C., washed with PBS and re-suspended in staining solution (50 μg/ml PI (Sigma), 200 μg/ml RNase A (Roche) for flow analysis. All flow cytometry data were collected using a Coulter EPICS XL-MCL Cytometer (Beckman Coulter) or a BD LSR I Cytometer (Becton Dickinson). Data were analyzed using FACScan (Becton Dickinson) and the WinMDI (J. Trotter, Scripps Institute) software packages.

### 7. Measurement of cAMP Levels

Cultured HepG2 cells that were approximately 70% confluent were treated with 200 μM mimosine for 28 hours. The mimosine-treated cells were then recovered in the regular culture medium for 2 hours before the treatment with the vehicle solution, 1 μM Triptonide, or 50 nM Trypsin in serum-free 1640 basal medium. Cells were harvested at various time points after the treatments. For one set of samples, the cells were treated with 1 μM Triptonide after they had recovered in the regular culture medium for 4 hours. The concentrations of cAMP in individual samples were then determined using a cAMP ELLISA Kit (CELL BIOLABS, Cat. No. STA-501) according to the protocol provided by the manufacturer.

### 8. Measurement of PKA Activity

Cultured HepG2 cells that were approximately 70% confluent were treated with 200 μM mimosine for 28 hours. The mimosine-treated cells were recovered in regular culture medium for 2 hours before the treatment with the vehicle solution or 1 μM Triptonide. To measure the acute effects of the treatments on PKA activities, the treatments were carried out in serum-free 1640 basal medium. Cells were harvested at various time points after the treatments. To measure the long-term effects of the treatments, the treatments were applied in regular culture medium for one hour. After the treatments, cells were returned to the regular culture condition and then harvested at various time points after the treatments. The levels of PKA activity of individual samples were determined by using the PepTag® Non-Radioactive cAMP-Dependent Protein Kinase Assay System (Promega, Cat. No. V5340) according to the protocol provided by the manufacturer.

### 9. Other Reagents

PKI-14-22 amide (myristoylated) was purchased from Tocris; and Vidarabine was purchased from Selleck. All other chemicals were purchased from Sigma-Aldrich, unless specified otherwise.

### Example 1

### Triptonide Can Exhibit a Cancer Cell-Specific Growth Inhibitory Effect

We devised a screening scheme in which both primary mouse hepatocytes (PMHs, representing non-cancer cells) and HepG2 cells (HCC cancer cells) are exposed to individual testing agents for one hour only and search for the agents that exhibit a significant growth inhibitory effect for the HepG2 cells but not for the PMHs. The one-hour exposure is intended to simulate the transient high concentrations within the liver as the results of either the first pass effects of some orally administered drugs, or of locally delivered drugs, anticipating that such a short term exposure is nevertheless sufficient to affect certain cell surface receptors without causing any significant off-target effects due to intracellular absorption. Such a strategy would also reduce false hits due to reversible cell cycle arrests. The IncuCyte Zoom system (Essen BioScience, Ann Arbor, Mich.) was used to determine the IC50 values for the short-term (one hour) exposure (IC50) for each tested compound, and to record the dynamic change of individual cells by taking photograph of the cultured cells every 2 minutes over the course of the experiment.

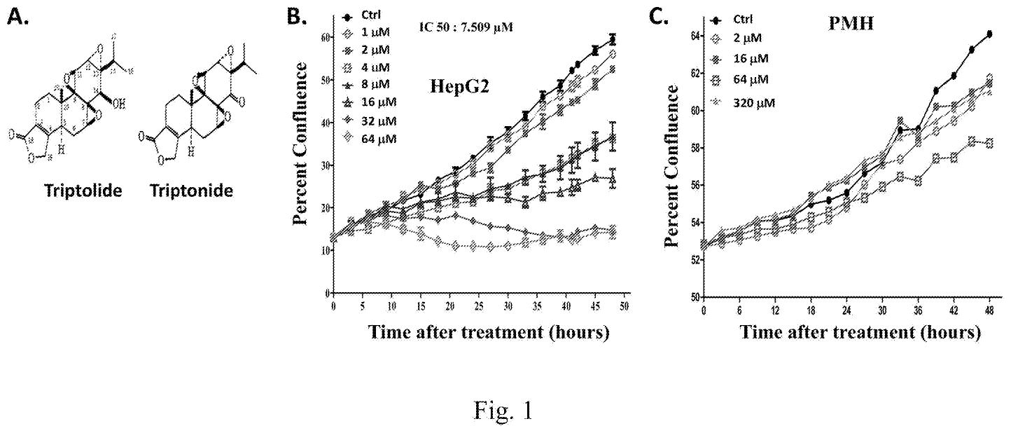

Exposure of HepG2 cells to Triptonide (FIG. 1A) at concentrations of 4 μM or higher for one hour caused a significant growth inhibition of HepG2 (IC50: 7.5 μM, FIG. 1B). Triptonide at 16 μM (slightly above the IC50) and above led to an apparent reduction in cell density, indicative of cell loss (cell death) being a contributing factor for the growth inhibitory effect (FIG. 1B). In contrast, the same treatments did not have a significant impact on the growth of PMHs (FIG. 1C). Remarkably, Triptonide at a concentration as high as 320 μM (about 25 folds higher than the IC50 for HepG2) had no significant growth inhibitory effect on the PMHs (FIG. 1C). These data, therefore, qualified Triptonide as the first candidate that has a specific growth inhibitory effect on HCC cancer cells but not its non-cancer counterpart (the PMHs), when applied as a one-hour treatment. Interestingly, continuous exposure of HepG2 and PMHs to Triptonide at concentrations above 200 nM caused complete death of HepG2 and PMHs (data not shown), revealing that the cancer cell-specific growth inhibitory effect of Triptonide is dependent on the short duration of exposure.

Under the one-hour drug exposure condition, the growth inhibition could be a direct reflection of the effect on irreversible growth inhibition (senescence), cell death, or both. Initially, the assay was carried out with mixed populations of cells at various stages of the cell cycle. Given that the cell cycle status of individual cells could affect their responses toward growth inhibitory and/or cell killing agents and that only a portion of cells were killed with the treatment of 16 μM Triptonide, we then asked whether mitogenic activation and/or the stage(s) of cell cycle progression could affect HepG2's response toward the treatments. In other words, does Triptonide exert its growth inhibitory effect by targeting the proliferating subpopulation of the cancer cell? To address this question, we carried out additional studies using cell populations that were enriched with cells in specific cell cycle stages. We used two different methods to obtain HepG2 cell populations that were enriched at various stages of the cell cycle. Specifically, serum starvation was used to obtain cell populations that are enriched in G0 and G1 phase of the cell cycle, and mimosine treatment was used to block cell progression at the late G1. Moreover, populations that were enriched for various stages of G1, S, G2 and M phase were then derived from these cell populations by allowing them to recover for certain time periods in the culture medium. The stages of the individual populations were determined by the 5-ethynyl-2′-deoxyuridine (Edu) incorporation experiment. In the Edu incorporation experiment, the cells are fed with Edu, an artificial building block for DNA, for a short period of time (about 30 minutes). The cells that are undergoing DNA synthesis (i.e. in the S phase) are the only cells that could incorporate Edu into their DNA. Thus, the ability of incorporating Edu serves as a marker for S phase cells.

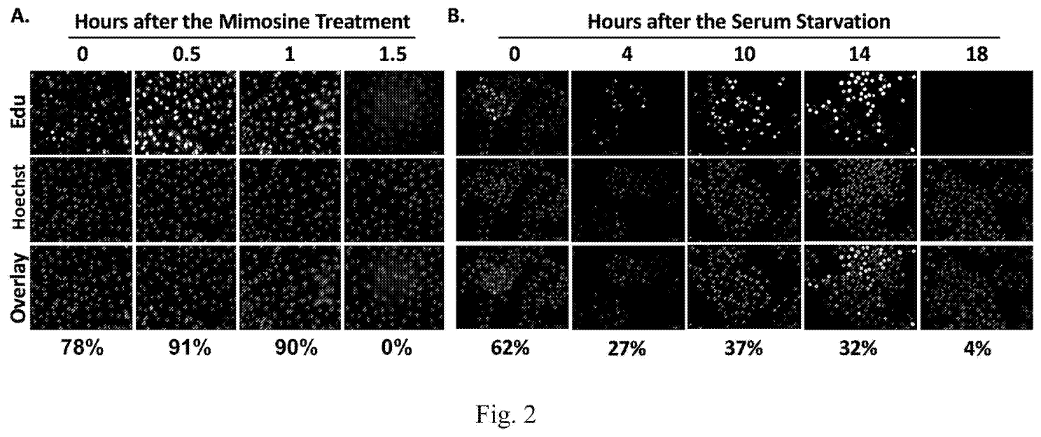

Edu incorporation experiment revealed that seventy-eight percent of the mimosine treated HepG2 cells incorporated Edu within 30 minutes. The majority of them (>90%) completed the S phase to reach the G2 phase within the next two hours (1.5 hours plus 0.5 hours for pulse labeling, FIG. 2A), confirming that majority of them were in fact at the G1/S boundary of the cell cycle. For the HepG2 cells that are subjected to the 48-hour serum starvation, 62% could incorporate Edu within the first 30 minutes after being released into the regular culture medium (with 10% serum) (FIG. 2B, 0, 4 hours). Given that cells could not enter S phase during serum starvation, this indicates that this 62% Edu incorporating cells were in the late G1 phase after the serum starvation but managed to progress into the S phase during the 30-minute exposure to the Edu. A second wave of approximately 37% EdU incorporating cells appeared at about 10 hours later (FIG. 2B, 10, 14, 18 hours). These were presumably those that were in the G0 or early G1 phase of the cell cycle following the serum starvation. Thus, the serum starvation led to the accumulation of cells in the G0/early G1 phase (37%) and the late G1 phase (62%), respectively.

Subsequently, an experiment was carried out to assess whether the effects of Triptonide on the serum starved cells were concentration- and/or time-dependent. Specifically, the serum starved cells were subjected to a one-hour treatment of Triptonide at various concentrations (from 0.1 to 10 μM), starting from their release from the serum starvation treatment (G0/early G1 or late G1) to up to 10 hour later (when the second wave of Edu incorporation occurred). We found that the one-hour treatments with Triptonide at concentrations below 1 μM had no significant impacts on the growth of these serum starved HepG2 cells, regardless when the Triptonide treatment was administered. All treatments with 10 μM Triptonide were growth inhibitory. Interestingly, at 1 μM, the treatment was growth inhibitory only when it was administered at the 0 and 2 hours after the starvation (data not shown).

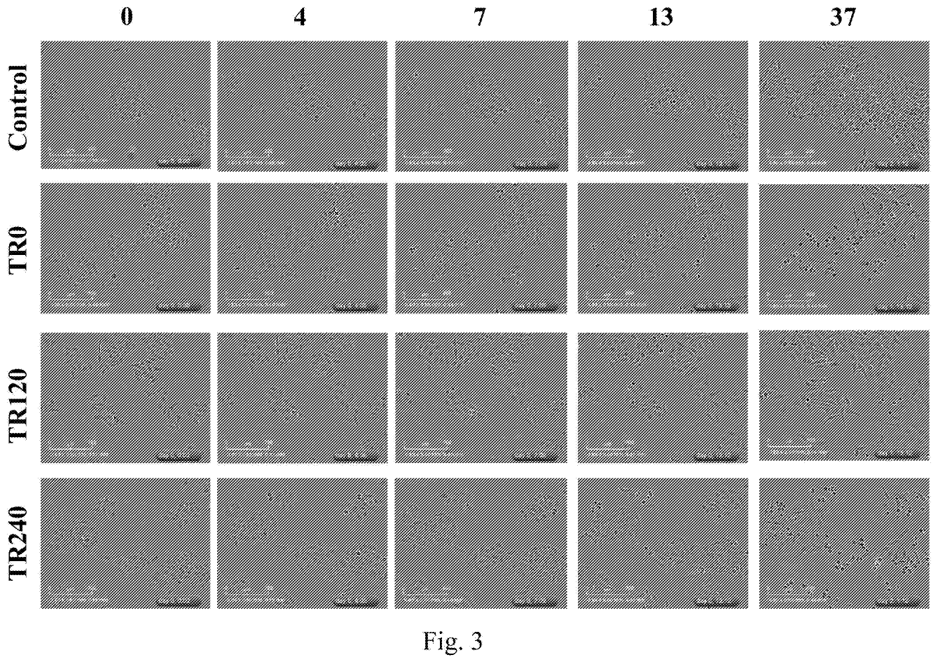

A more detailed study was carried out to examine the responses when the 1 μM Triptonide treatment was applied at 0, 1, 2, 4 hours after the serum starvation. The data showed that after being released into regular culture medium, some of the serum starved HepG2 cells (presumably the G1 subpopulation) began entering mitosis at hour 13 and reached a peak at hour 16 (FIG. 3, Control-13). This first wave of mitosis was apparently coming from the late G1 subpopulation, as cells of the G0 subpopulation were still at the G1 or S phase at this point (FIG. 2B). By hour 37, the cell number of the control was more than two-folds of the starting number (FIG. 3, Control-0, 37). These data showed that most of these serum starved cells (both the G0/early G1 and the late G1 subpopulations) were able to resume the cell cycle progression, and that the late G1 subpopulation began entering mitosis about 13 hours after returning back to the culture medium and were capable of undergoing productive mitosis.

The 1 μM Triptonide treatment caused a significant accumulation of cells with a spherical morphology and condensed chromatin only when the treatment was administered at 0 and 4 hours after the serum starved cells were cultured in regular medium. The time of on-set for the accumulation of the cells with the unique spherical morphology and condensed chromatin became quite evident by hour 13 (FIG. 3, TR0-13, TR240-13), coinciding with the time when the control cells began entering mitosis (FIG. 3, Control-13, and data not shown). Importantly, such cells that had the spherical morphology and condensed chromatin, once occurred, remained largely unchanged for up to 37 hours (FIG. 3, TR0-37, TR240-37), which is indicative of mitotic catastrophe. Thus, the data clearly indicate that Triptonide causes a previously unknown effect: the induction of mitotic catastrophe. Moreover, cumulatively, approximately 60% of the cells exhibited the features of the mitotic catastrophe (FIG. 3, TR0, TR240), revealing that the Triptonide treatment caused the late G1 subpopulation (62%) (FIG. 2B) to undergo mitotic catastrophe (The cells that exhibited such features are therefore referred to as mitotic catastrophe cells herein). Thus, overall, these data indicate that the late G1 cells were sensitive to the treatment at only 0 and 4 hours after the release from the starvation, when they were at the points around the G1/S and the S/G2 transition, respectively, according to the data from the Edu-incorporating experiments (FIG. 2B). Meanwhile, the Triptonide treatment, when applied at 0 to 4 hours after the release from the serum starvation, did not appear to cause a significant mitotic catastrophe-inducing effect on the remaining 37% of cells that were at the G0 or early G1 phases, which traversed the S phase between 10 to 14 hours after releasing from the starvation (FIG. 2B) and should remain as G0/early G1 at 0 and 4 hours after the release from the serum starvation. Thus, these data also revealed that HepG2 at the late G1 to early S phase and the late S to early G2 (referred to at around the G1/S and S/G2 transition hereafter) were uniquely sensitive to a mitotic catastrophe-inducing effect of Triptonide, but quiescent (G0) and early G1 HepG2 cells were not sensitive to such an effect.

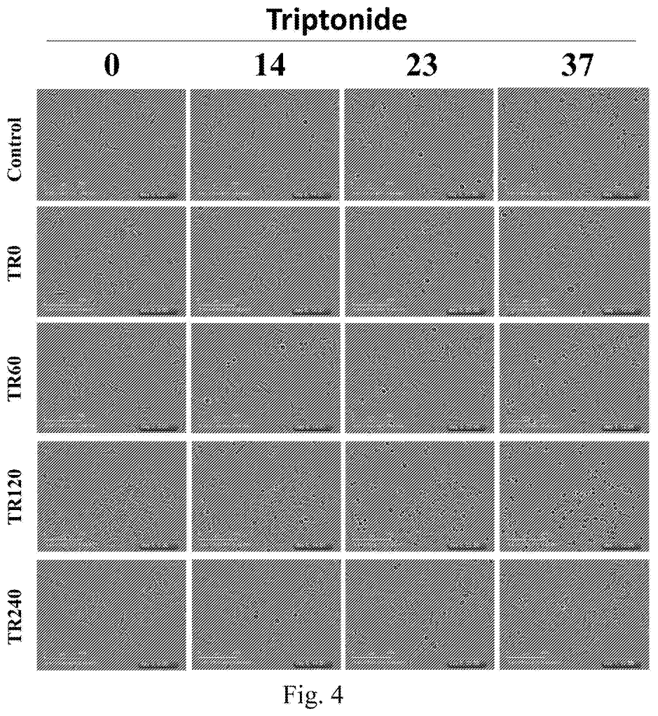

A similar series of experiments were then carried out with the mimosine-treated cells in order to rule out potential effects that were specific to serum starvation and to examine whether any other points during the cell cycle when the cells were sensitive to the Triptonide treatment. The mimosine-treated HepG2 cells could also resume cell cycle progression. Of note, a significant fraction of them have entered mitosis at hour 11 and the percentage of mitotic figure peaked at about hour 14 after the release from the mimosine treatment (FIG. 4, Control-14 and data not shown). Together with the data from the Edu incorporation experiments, these data showed that these mimosine-treated HepG2 cells (at the G1 phase) progressed through the S phase and enter G2 relatively synchronously. However, they became rather asynchronous thereafter and reached metaphase over a period of many hours (FIG. 4, Control 14, 23, and 37 hours). A preliminary experiment was carried out with the 1 μM Triptonide treatment from hour 0 to hour 14 (when the first wave of cells had completed mitosis). The result from the preliminary experiment showed that the cells were only sensitive when the treatment was applied at 2 hours after the cells were on the mimosine-free medium. A more detailed follow-up study confirmed that the time around the S/G2 transition is the only point when the mimosine-treated HepG2 cells were sensitive to the mitotic catastrophe-inducing effect of the Triptonide treatment (FIG. 4A, TR120). Also, in this case, the mitotic catastrophe cells persisted throughout the course of the experiment (up to 23 hours), and the time of onset for the accumulation of the cells became quite evident by hour 11 and peaked at hour 14 after the mimosine treatment (FIG. 4, TR120-14 and data not shown), coinciding with the time when the cells began entering mitosis and the mitotic index reached its peak value in the control (FIG. 4, Control-14, and TR240-14). Interestingly, when the treatment was applied at hour 0 after the release, the cells became static, but no significant accumulation of mitotic catastrophe cells were observed (FIG. 4, TR0). When the same treatment was administered at other time points, for example, at 60 and 240 minutes, after the mimosine release, no significant increases in mitotic catastrophe cells were observed (FIG. 4, TR60, TR240).

Thus, the combined data from experiments using HepG2 cells that were treated using two different methods show that HepG2 are sensitive to a unique mitotic catastrophe-inducing effect of Triptonide in a cell cycle stage-specific fashion, namely around S/G2 transition points. A sensitive point around the G1/S transition was detected when serum starved HepG2 cells (at (G0/early G1 plus late G1) were used, but not when mimosine-treated cells (at the G1/S boundary) were used. Rather, the mimosine-treated G1/S cells became static following the Triptonide treatment, which is expected to prevent the cells to progress into mitosis or mitotic catastrophe. Thus, the mimosine-treated cells are not suitable for assessing whether Triptonide exerts a mitotic catastrophe-inducing effect around the G1/S transition point. Accordingly, it appears likely that Triptonide can exert a mitotic catastrophe-inducing effect around G1/S transition point. Importantly, quiescent (G0) cells were refractory to the mitotic catastrophe-inducing effect of Triptonide.

### Example 2

### Triptonide and Triptolide Exhibit Distinctive Effects on HepG2 Cells

In this example, the effects of Triptonide and Triptolide on HepG2 cells were compared.

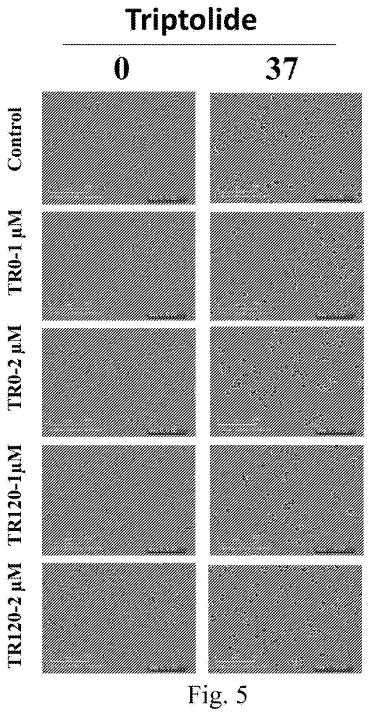

A one-hour treatment of the mimosine-treated HepG2 cells with 1 μM Triptolide was growth inhibitory, but did not cause mitotic catastrophe, regardless when it was administered (FIG. 5, TR120, TR0-1 μM, TR120-1 μ). However, a one-hour treatment with 2 μM Triptolide was not only growth inhibitory, but also caused chromatin condensation when administered both immediately after the mimosine treatment, and 2 hours after the treatment, albeit to a lesser extent (FIG. 5, TR0-2 μM, TR120-2 μM, and data not shown).

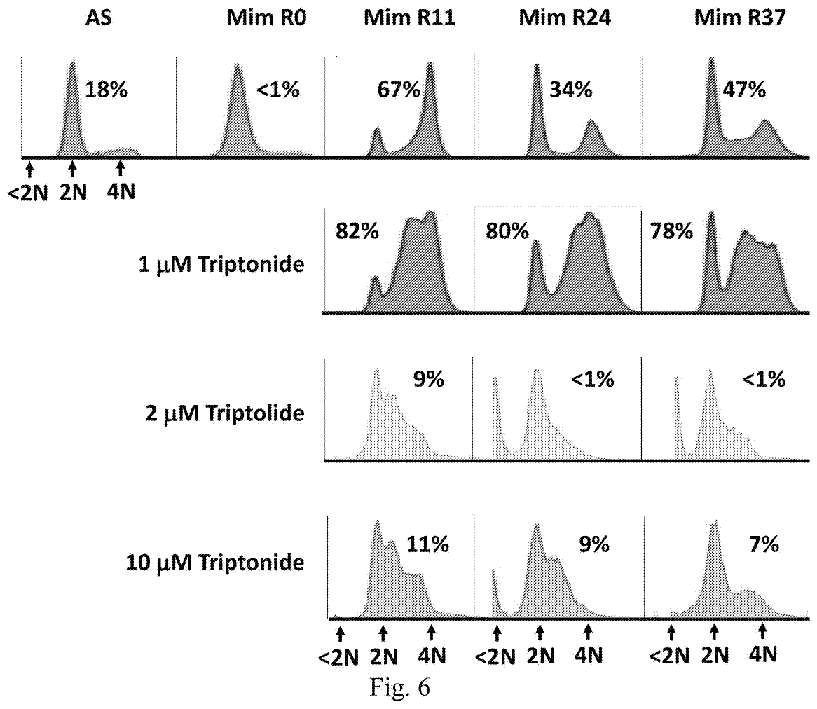

Cell cycle progression analyses showed that the mimosine treated HepG2 cells were able to resume productive cell cycle progression. The majority of them reached the G2/M phase with a near 4N DNA content at 11 hours after the mimosine release (Mim R11). Meanwhile, those mimosine treated cells that were additionally treated with 1 μM Triptonide exhibited a significant accumulation of G2/M cells (82%, 80%, and 78% at 11, 24, and 37 hours, respectively). These data further validate the conclusion that the spherical cells that occurred in the mimosine-treated cells 2 hours after the mimosine treatment were indeed the result of mitotic catastrophe. In contrast, cells that were treated with either 2 μM Triptolide or 10 μM Triptonide were first arrested at the G1, S, and G2 phases at 11 hours (Mim R11). By 24 hours (Mim R24), the percentages of S and G2 diminished, while significant peaks of the sub-G1 (<2N, apoptotic) population became evident for both. Importantly, there were no accumulations of cells with a 4N DNA content, revealing the lack of a significant mitotic catastrophe effect (FIG. 6). These data, taken together with those from live cell imaging, therefore revealed that the cells treated with 1 μM Triptonide were able to enter the G2/M phase but then failed to complete mitosis. Instead, they underwent mitotic catastrophe. Meanwhile, cells treated with 1 μM, 2 μM or 10 μM became arrested at G1 (2N) or underwent apoptosis as S or G2 cells. Therefore, these data indicated that despite both Triptonide and Triptolide could have significant growth inhibitory effects at the low μM levels, they are distinct with respect to their potencies as well as the underlying mechanisms. Specifically, while Triptonide could cause only G2/M arrest/mitotic catastrophe, Triptolide primarily caused G1 arrest and apoptosis, but not mitotic catastrophe.

### Example 3

### Triptonide Targets PAR2 to Induce Mitotic Catastrophe

In order to identify the target(s) of Triptonide, we examined whether the growth inhibitory effect of Triptonide was dependent on PAR2.

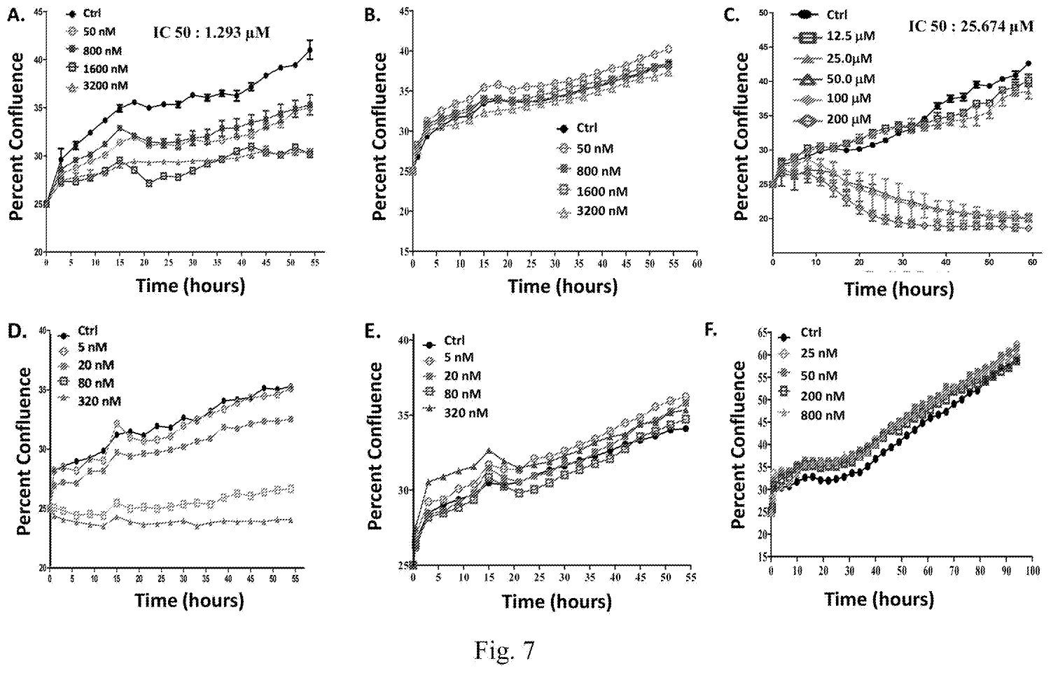

To address this question, we took a genetic approach. Specifically, since human PAR2 and its mouse homologue Par2 are highly conserved and primary keratinocytes express Par2 (intriguingly, only the differentiated postmitotic keratinocytes express high levels of Par2 in vivo), we decided to examine whether Triptonide could cause cell death in a Par2-dependent manner by comparing the Triptonide sensitivities between primary keratinocytes derived from wild-type and Par2 knockout mice, respectively. We found that 50 nM of Triptonide (the lowest dose tested) was effective in inhibiting the growth of wild-type keratinocytes, whereas 1.6 μM was sufficient to cause a complete growth inhibition (IC50: 1.293 μM) (FIG. 7A). In contrast, for the Par2 knockout keratinocytes, Triptonide at concentrations up to 26 μM did not cause significant growth inhibition (IC50: 25.674 μM) (FIG. 7B, 7C). When administered via the conventional continuous exposure manner, Triptonide still exhibited a more potent growth inhibitory effect on the Par2 wild-type PMHs than their Par2 knockout counterparts (FIG. 7D, 7E). These data demonstrated that Triptonide had a Par2-dependent growth inhibitory effect on primary keratinocytes at a concentration range between 50 nM and 26 μM when applied via the one-hour treatment scheme.

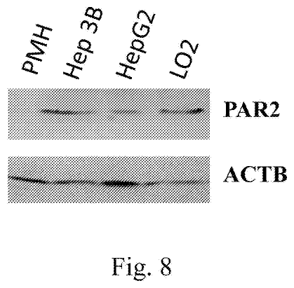

Treatment of wild-type keratinocytes with Trypsin, a cognate activator of Par2/PAR2, did not cause any significant cell death (FIG. 6F), consistent with the expectation that the normal activation of the PAR2-dependent pathway per se was not cytotoxic in nature. These data have strongly suggested that an abnormal activation, rather than the canonical activation of the PAR2/Par2 pathway, is likely the underlying basis for the Triptonide-mediated Par2/PAR2-dependent effects on cell death. Also, since our initial finding that primary mouse hepatocytes were not sensitive to the growth inhibitory effect of Triptonide, these data also implied that primary mouse hepatocytes (PMHs) were either deficient in Par2 or expressed the Par2 that were not conserved with its human counterpart. Western blot experiment revealed that PMHs were in fact deficient in Par2 (FIG. 8). These data in combination with the highly conserved nature between the mouse Par2 and PAR2 (its human counterpart) support that the Triptonide-induced growth inhibitory effect, and accordingly, the mitotic catastrophe-inducing effect, is Par2-dependent in mouse cells and PAR2-dependent in human cells, respectively.

### Example 4

### Triptolide Was Not as Effective as Triptonide in Causing Par2-Dependent Death of PMKs

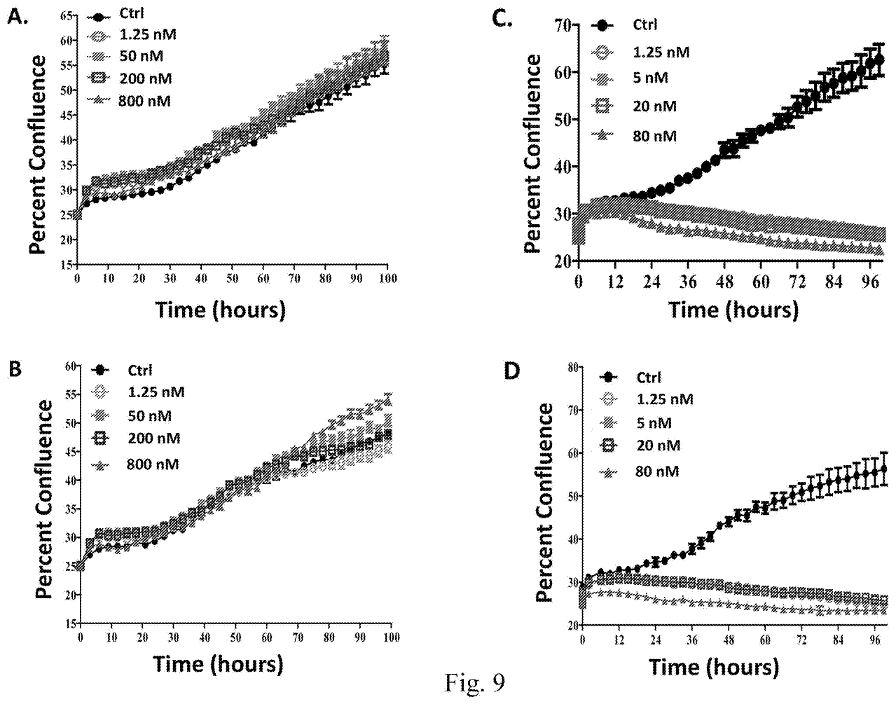

Interestingly, while Triptonide exhibited a significant growth suppressive effect on the wild-type keratinocytes at as low as 50 nM, its close relative Triptolide did not have any significant growth inhibitory effect on either the wild-type or the Par2 knockout keratinocytes even at concentration as high as 800 nM (FIG. 9A, 9B), indicating that Triptolide is either less potent than Triptonide, or not effective at all in causing the Par2-dependent cell death. In stark contrast, however, when exposed continuously, Triptolide at a concentration as low as 1.25 nM (the lowest concentration examined) resulted in a complete lethality for both wild-type and the Par2 knockout keratinocytes (FIG. 9C, 9D). In comparison, Triptonide at concentrations as high as 320 nM had no significant toxic effect on PMHs (FIG. 9D). These data have provided additional evidence to support the distinct effects of Triptolide and Triptonide. That is, when administered in a one-hour scheme, Triptonide, but not Triptolide, is effective in causing the unique Par2/PAR2-dependent cell death on mitogenically activated cells; and that Triptolide is a very potent poison while Triptonide was not toxic when it is applied continuously at concentrations between 1.25 nM and 320 nM.

Also, it became apparent that the mitotic catastrophe-inducing effect of the Triptonide treatment manifested itself in a delayed fashion. Specifically, based on the data from our Edu incorporation experiment, it took at least 11 hours for mimosine-treated cells to traverse from the G1/S boundary (one of the sensitive point) to the G2/M transition point, when the mitotic catastrophe began to occur (FIGS. 3 and 4). Thus, there exists a gap of at least 8 hours (taking into account of the one-hour treatment time) from the time of the Triptonide treatment (at 2 hours after the mimosine treatment) to the onset of the mitotic catastrophe phenotype (at 11 hours after the mimosine treatment). To the best of our knowledge, there have not been any reports of any agents that could cause such a unique effect. Thus, we discovered a new way for the targeted killing of cancer cells and Triptonide represented the first agent that could be used to implement such unique type of targeted cancer cell killing. It then became crucial to identify the target pathway through which Triptonide induces such a unique effect in exploiting this new type of cancer cell-specific killing for the development of new targeted anticancer therapies.

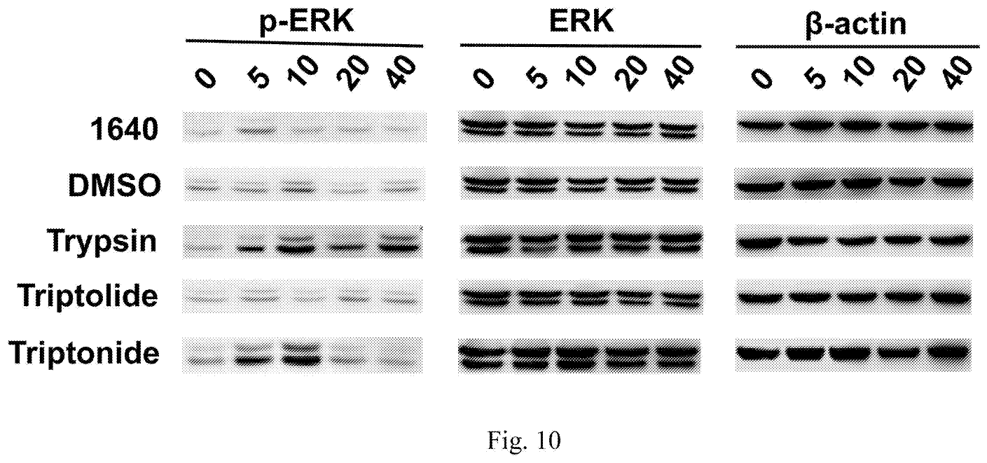

Given the Par2/PAR2-dependent nature of the mitotic catastrophe-inducing effect of Triptonide, activation of Par2/PAR2-mediated signaling then became a candidate for causing such an effect. Canonical activation of Par2/PAR2 by trypsin leads to the activation of the Gq-coupled pathway, and indirectly the activation of the MAPK-ERK pathway. Alternatively, Par2/PAR2 activation has also been reported to result in the activation of the Gs-coupled pathway, namely the Gs-Adenylyl cyclase (AC)-cAMP-PKA pathway. Because the MAPK-ERK pathway had been implicated to have important roles during the cell cycle, including G2 and mitosis, and Triptolide had been identified as a potent Par2/PAR2 antagonist, we asked whether Triptonide could cause the unique mitotic catastrophe-inducing effect by perturbing the proper action of the MAPK-ERK pathway. To test this possibility, we examined whether Triptonide could function like Triptolide as a potent Par2/PAR2 antagonist. As a hallmark of Par2/PAR2 activation is the unique two-stage ERK phosphorylation (an acute phase of increase followed by a delayed beta arrestin-dependent increase), we examined the effect of Trypsin (a cognate PAR2 agonist), Triptonide, and Triptolide on ERK phosphorylation in HepG2 cells. We found that, as expected, Trypsin caused a two-stage increase in the levels of phosphorylated ERK in serum-starved G0/G1 HepG2 cells. Triptolide, a Par2/PAR2 antagonist, had no significant effects on the levels of phosphorylated ERK. To our surprise, however, Triptonide caused only an acute transient increase at the levels of phosphorylated ERK (FIG. 10). Thus, assuming that this effect of Triptonide on ERK phosphorylation was Par2/PAR2-mediated, it would appear that Triptonide functions as an unusual agonist, rather than antagonist for Par2/PAR2. It follows then that Triptonide does not cause the mitotic catastrophe-inducing effect by acting as a Par2/PAR2 antagonist. Such a conclusion is consistent with the fact that although some cancer cells are apparently “addicted” to Par2/PAR2 for growth and/or survival and consequently inhibition of Par2/PAR2 could cause growth inhibition, Par2/PAR2 is not required for growth and survival of any cell types. Moreover, given the role of MAPK-ERK in promoting proliferation, the ability of Triptonide to activate ERK phosphorylation implies that it should have a proliferation enhancing effect instead. Accordingly, another type or types of Triptonide-induced Par2/PAR2-mediated effects should be responsible for the unique mitotic catastrophe-inducing effect on Par2/PAR2-expressing cells.

### Example 5

### Triptonide Represents the Very First Small Molecule Biased Agonist for the Gαs-Coupled PAR2 Signaling Pathway

Elevation of the PKA activity in the cytoplasm have proven an effective mechanism for suppressing G2/M transition in both mammalian oocytes and somatic cells, while prolonged or permanent inhibition of G2/M transition could lead to mitotic catastrophe. Since PAR2 activation can be coupled to the activation of the Gαs-AC-cAMP-PKA pathway, i.e. the elevation of PKA activity, we then considered the hypothesis that Triptonide causes the mitotic catastrophe-inducing effect by activating the Gαs-coupled pathway through PAR2, i.e., through the elevation of cytoplasmic activity of PKA by activating the AC-cAMP-PKA pathway. This hypothesis led to several predictions: 1) CDK1 activation would not occur prior to the time of the G2/M transition; 2) the AC-cAMP-PKA pathway would be activated; 3) the level of cytoplasmic PKA would remain elevated around the time of the G2/M transition; 4) the growth inhibitory and mitotic catastrophe-inducing effect of Triptonide would be dependent on the AC-cAMP-PKA pathway. We then carried out the following experiments to examine the validity of these predictions.

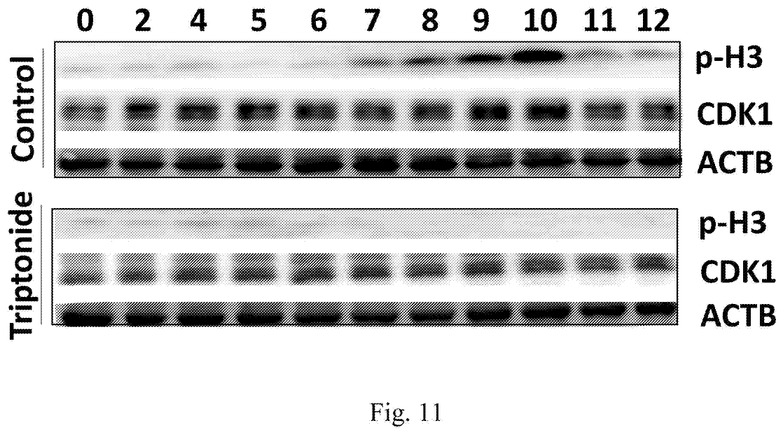

First, we compared the kinetics of CDK1 activation between untreated and Triptonide-treated HepG2 cells, which were mimosine-synchronized, as they progressed through the S, G2 phases into mitosis (hour 0 to hour 12). Specifically, we assessed the activity of CDK1 by monitoring the phosphorylation of histone H3 by Western blot. The results of this experiment showed that in the mimosine-synchronized HepG2 cells, the level of phosphorylated histone H3 (p-H3) increased dramatically at hour 10 and then decreased rapidly with the next hour (FIG. 11) (a dramatic increase of CDK1 activity at 10 is consistent with a dramatic rise in mitotic index at hour 11 (FIG. 4 and data not shown). In contrast, the level of histone H3 phosphorylation remained very low in Triptonide-treated cells throughout the entire monitored period (FIG. 11). Thus, the data indicate that CDK1 was not activated in the cells that were treated with 1 μM Triptonide at a time when they were sensitive to the Triptonide, despite the vast majority of these cells had acquired a 4N DNA content and presumably entered G2 (FIG. 6). These data, therefore, support our hypothesis that Triptonide causes the mitotic catastrophe-inducing effect by activating the Gαs-coupled pathway through PAR2, i.e., through the elevation of cytoplasmic activity of PKA.

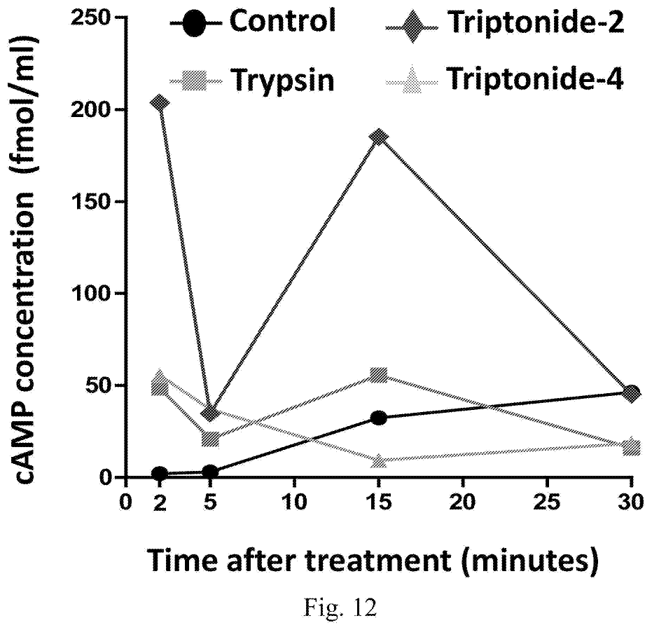

Next, we examined whether the Triptonide treatment could indeed result in the activation of the AC-cAMP-PKA pathway. Specifically, we examined the effects of the Triptonide treatment on the levels of cAMP and PKA activity. In this case, the experiments were carried out in a serum-free medium in order to minimize the influence of the levels of cAMP by the serum. Mimosine-synchronized cells were subjected to the Triptonide treatment at both a sensitive and a non-sensitive time (hour 2 and 4 after the mimosine treatment in FIG. 4, respectively). Also, a Trypsin-treatment and a Triptonide treatment administered at a non-sensitive time (hour 4 after the mimosine treatment) were included as controls. We found that Trypsin caused modest and transient elevations in levels of cAMP. The Triptonide treatment administered at the non-sensitive time (4 hours after the mimosine treatment) resulted in a modest elevation in the levels of cAMP, with amplitudes similar to that caused by the Trypsin treatment. In contrast, the Triptonide treatment at 2 hours after the mimosine treatment resulted in two waves of elevation in the levels of cAMP, both with much higher amplitudes (about 4 folds higher than that of the control) (FIG. 12). This unexpected finding has therefore revealed the unusual nature of the Triptonide-mediated activation of the AC-cAMP-PKA pathway: it results in more than one round of elevation in the levels of cAMP. Importantly, the effects were observed only when the Triptonide treatment was applied at 2 but not 4 hours after the mimosine release, linking these effects with the mitotic catastrophe-inducing effect.

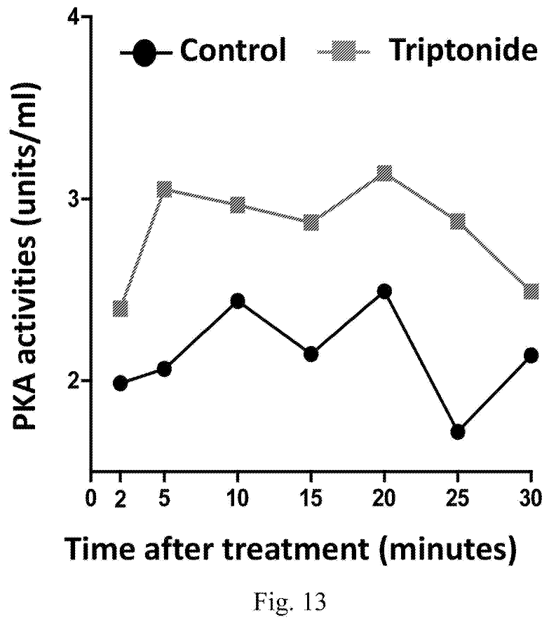

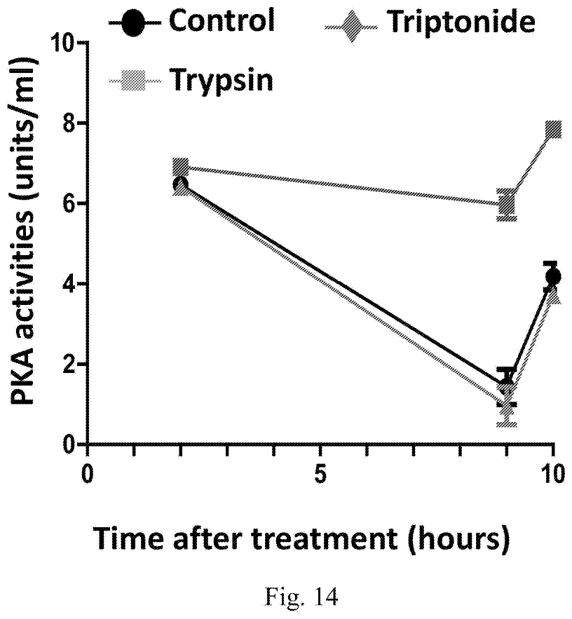

Importantly, however, in order to maintain the suppression of the G2/M transition, the effect of the Triptonide on CDK1 activity would need to be maintained until or possibly even beyond the point of the G2/M transition, which is supported by the data shown in FIG. 11. A possible scenario of achieving this effect would be that the activation of the AC-cAMP-PKA pathway by the initial one-hour Triptonide treatment could somehow lead to the establishment of an elevated level of PKA activity around the time of the G2/M transition and beyond. To examine this possibility, we tested the effect of the Triptonide treatment on PKA activity. First, we found that under a serum free condition, a two-wave elevation in the levels of PKA activities was also observed within the first 30 minutes after the Triptonide treatment (FIG. 13). The key question then was whether the treatment applied under the normal cell culture condition could lead to an elevated level of PKA activity around the time of the G2/M transition, i.e. at about 10 hours after the mimosine treatment. In this case, the mimosine-treated cells that were enriched in the G1/S boundary were allowed to recover in regular culture medium for up to 10 hours, just prior to the G2/M transition and when the level of CDK1 activity peaked in the control (FIG. 4, FIG. 11). We found that in control and Trypsin-treated cells, the level of PKA activity was reduced significantly at hour 9, just prior to the time of G2/M transition. In contrast, in Triptonide-treated cells, the levels of PKA activities remained largely unchanged at hour 9 and 10 (FIG. 14). Thus, a brief one-hour treatment with Triptonide around the S/G2 transition point led to an inability of the treated cells to reduce the level of their PKA activity many hours later as they were on schedule for the G2/M transition. Given the previous demonstration that elevation of PKA activity alone is sufficient to suppress G2/M transition during mitosis, this finding has provided a plausible explanation for the mitotic catastrophe-inducing effect of Triptonide and lent further support to our hypothesis that Triptonide causes the mitotic catastrophe-inducing effect, at least in part, by activating the AC-cAMP-PKA pathway.

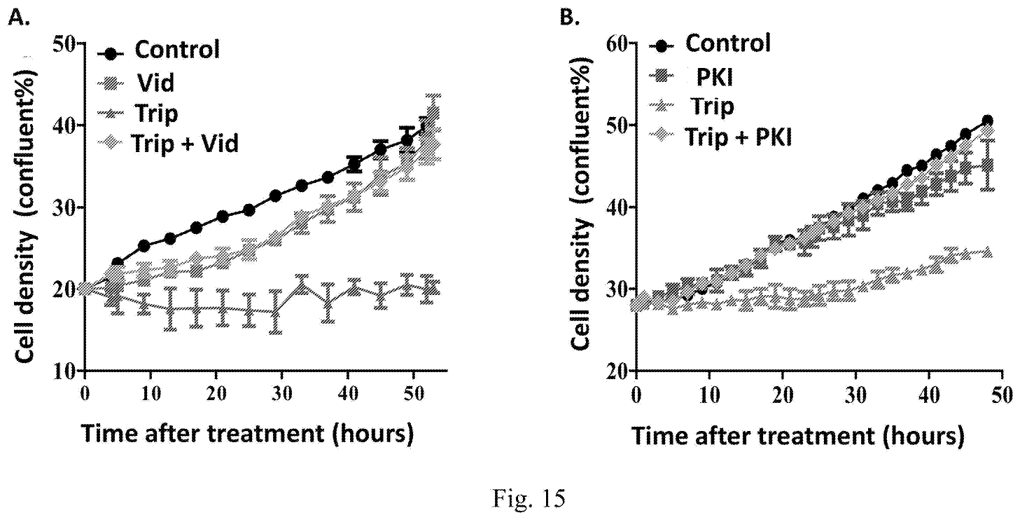

We next addressed the question regarding whether the growth inhibitory effect or mitotic catastrophe-inducing effect of Triptonide was in fact caused by the abnormal activation of the Gαs-AC-cAMP-PKA pathway. To address this question, we first treated the mimosine-synchronized HepG2 cells with Vidarabine (a small molecule inhibitor for adenylyl cyclase), Triptonide either alone or in combination and then examined their effects on the growth rate and/or mitotic catastrophe-inducing effect. We found that the treatment with Triptonide, but not those with Vidarabine, exhibited a significant growth inhibitory effect on the HepG2 cells. Meanwhile, the treatment with both Vidarabine and Triptonide also had no significant effects on the growth of the HepG2 cells (FIG. 15A).

We then carried out a similar set of experiments with myristoylated PKA inhibitor 14-22 amide, a cell permeable PKA inhibitor. We found that the treatment with the PKI inhibitor alone had no significant effect on the growth of the HepG2 cells, while the Triptonide treatment exhibited a significant growth inhibitory effect on these cells. Yet, co-treatment with the PKI inhibitor and the Triptonide did not cause any significant growth inhibitory effect on the HepG2 cells (FIG. 15B). Thus, inhibiting the AC-cAMP-PKA pathway with two different methods both resulted in the protection of the HepG2 cells from the growth inhibitory effect of the Triptonide treatment. These data, therefore, support the contention that the growth inhibitory and mitotic catastrophe-inducing effects of Triptonide on HepG2 cells are indeed caused by the abnormal activation of the AC-cAMP-PKA pathway.

Also, given that the growth inhibitory effect of Triptonide on moues primary keratinocytes was also dependent on Par2 (FIG. 7), it became clear that Triptonide exerts its growth inhibitory effect as well as the mitotic catastrophe-inducing effect on mouse and human cells by acting as an unusual agonist for Par2 and PAR2 via the activation of the Gαs-AC-cAMP-PKA pathway. To the best of our knowledge, this has also led to the identification of Triptonide as the first small molecule agonist for the PAR2-Gαs-AC-cAMP-PKA signaling pathway.

### Example 6

### The Majority of Cell Lines of Multiple Types of Human Cancers are Sensitive to Triptonide

Previously, it has been reported that PAR2 is expressed in many cancer cell lines and in a significant fraction of many tumor types examined. Thus, we then examined whether this novel anticancer paradigm could be exploited for the specific killing of cancer cells in many cases of HCCs and/or of other types of cancers.

To address this possibility, we investigated whether other cell lines, including other HCC cell lines, cell lines from other type of cancers, as well as non-cancer immortalized/transformed cell lines, were also sensitive to Triptonide. Specifically, a total of 51 human cancer cell lines, representing 10 types of human cancers (6 non-small cell lung cancer cell lines, 5 colon cancer cell lines, 5 central nerve system cancer cell lines, 8 Melanomas cell lines, 4 ovarian cancer cell lines, 4 renal cancer cell lines, 2 prostate cancer cell lines, 3 breast cancer cell lines, 7 hepatocellular carcinoma cell lines, and 4 gastric cancer cell lines) were analyzed. The IC50s for the 51 cancer cell lines range from 0.41 μM (NCI-H23, a non-small cell lung cancer line) to 51.039 μM (SNB-19, a central never system cancer line). The overall mean value was 7.308 μM, which was slightly lower than that of HepG2 (7.5 μM) (Table 1). Among them, 34 lines (66.7%), consisting of at least one representative from all of the 10 cancer types, have ID50 values lower than that of HepG2 (Table 1). Thus, the vast majority of these cell lines of 10 different cancer types are more sensitive to Triptonide than HepG2.

Taken together, these data demonstrate that the majority of cancer cell lines derived from 10 types of human cancers are very sensitive to the one-hour treatment with Triptonide. In particular, 66.7% of the cancer cell lines, with at least one representative from each of the 10 types of cancers examined, has IC50 values lower than that of HepG2, the model cell line that was used in the initial screen as well as many of the subsequent experiments, including the antitumor experiment to be described below.

TABLE 1 [symbol]
Growth Inhibitory Effects of [symbol] (One Hour Exposure)

| Cell Line | Tumor Type of Origin | IC₅₀ (uM) |
|---|---|---|
| EKVX | Non-Small Cell Lung | 2.383 |
| HOP-62 | Non-Small Cell Lung | 9.235 |
| NCI-H226 | Non-Small Cell Lung | 11.218 |
| NCI-H23 | Non-Small Cell Lung | 0.415 |
| NCI-H460 | Non-Small Cell Lung | 8.437 |
| NCI-H522 | Non-Small Cell Lung | 23.142 |
| HCC-2998 | Colon | 2.774 |
| HCT-116 | Colon | 2.572 |
| HCT-15 | Colon | 1.182 |
| HT29 | Colon | 1.696 |
| SW-620 | Colon | 11.642 |
| SF-268 | CNS | 11.797 |
| SF-295 | CNS | 14.559 |
| SF-539 | CNS | 1.143 |
| SNB-19 | CNS | 51.039 |
| U251 | CNS | 43.176 |
| LOX IMVI | Melanoma | 1.774 |
| MALME-3M | Melanoma | 3.437 |
| M14 | Melanoma | 4.934 |
| MDA-MB-435 | Melanoma | 1.913 |
| SK-MEL-2 | Melanoma | 0.508 |
| SK-MEL-28 | Melanoma | 17.834 |
| UACC-257 | Melanoma | 16.002 |
| UACC-62 | Melanoma | 9.350 |
| IGR-OVI | Ovarian | 4.686 |
| OVCAR-3 | Ovarian | 4.903 |
| OVCAR-4 | Ovarian | 1.539 |
| OVCAR-5 | Ovarian | 8.229 |
| OVCAR-8 | Ovarian | 7.295 |
| NCI/ADR-RES | Ovarian | 8.335 |
| SK-OV-3 | Ovarian | 3.887 |
| A498 | Renal | 4.337 |
| ACHN | Renal | 14.997 |
| CAKI-1 | Renal | 6.146 |
| SN12C | Renal | 1.066 |
| PC-3 | Prostate | 6.707 |
| DU-145 | Prostate | 2.376 |
| MCF7 | Breast | 0.592 |
| HS 578T | Breast | 1.858 |
| T-47D | Breast | 1.809 |
| HepG₃ | Hepatocellular carcinoma | 7.509 |
| Hep3B | Hepatocellular carcinoma | 1.281 |
| SNU-449 | Hepatocellular carcinoma | 4.917 |
| SNU-387 | Hepatocellular carcinoma | 6.817 |
| Huh7 | Hepatocellular carcinoma | 1.745 |
| LM9 | Hepatocellular carcinoma | 4.239 |
| SMMC7721 | Hepatocellular carcinoma | 3.910 |
| SGC?901 | Gastric cancer | 11.977 |
| MGC803 | Gastric cancer | 2.580 |
| AGS | Gastric cancer | 4.065 |
| BGC823 | Gastric cancer | 10.366 |
| Mean Value | | 7.655 |
| GES-1 | | 16.719 |
| LO2 | | 0.642 |

*, **: Immortalized cell lines derived from gastric epithelium and hepatocytes respectively.

### Example 7

### Transformed Non-Cancer Cells were PAR2 Positive and Sensitive to Triptonide

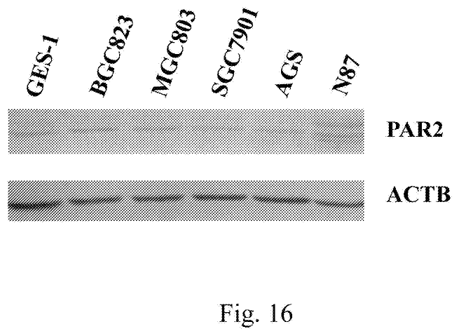

In the experiment described above, two immortalized cell lines (LO2, immortalized hepatocytes; and GES-1, immortalized gastric epithelium) were also included as the non-cancer control cell lines. Remarkably, we noticed that both of these two immortalized cell lines examined were sensitive to Triptonide (Table 1). This finding prompted us to hypothesize that PAR2 was expressed in these non-cancer cells, or in other words, PAR2 activation could constitute a common early event (or the so-called “driver event”) of oncogenesis (and hence a desirable target for anticancer drug development). Indeed, we found that PAR2 was clearly expressed not only in HCC cell lines (FIG. 8) and gastric cancer cell lines (FIG. 16), but also in both immortalized cell lines examined (FIG. 8, FIG. 16). And as expected, Par2 was not detectable in extracts of primary mouse hepatocytes (FIG. 8), which were resistant to the Triptonide treatment (FIG. 1). Thus, these data clearly show that PAR2 is activated in immortalized cell lines. Since immortalization constitutes an early event in cellular transformation and oncogenesis, this has also raised the possibility that PAR2 activation is an early event in oncogenesis for some cancers. In addition, the data confirm a correlation between the sensitivity to Triptonide and Par2/PAR2 expression in mouse and human cells.

### Example 8

### Triptonide Exhibited a Potent Antitumor Activity In Vivo

As discussed earlier, the unique Triptonide-mediated, PAR2-dependent killing of proliferating cells in the context of the restricted expression of PAR2 in non-dividing cells in vivo prompted us to further evaluate the feasibility of using Triptonide and/or similar PAR2 ligands to instigate a targeted anticancer therapy for PAR2-expressing cancers. However, given that long-term exposure to Triptonide were quite toxic to the Par2-expressing primary mouse keratinocytes at concentrations as low as 50 nM (FIG. 7), the implementation of a short-term exposure, i.e. short duration of the desired plasma concentration, would be imperative in order to target the PAR2-dependent killing to cycling cancer cells while avoiding significant non-specific toxicities toward PAR2-expressing non-cancer cells. In this regards, it was fortunate that Triptonide appears to exhibit a very rapid redistribution kinetics in rodent which is associated with several dozen folds of decrease in its plasma concentration following an oral dosing. Thus, we decided to evaluate the potential antitumor activity of Triptonide. Specifically, since HepG2 cells exhibited a level of sensitivity toward Triptonide that is lower than 66.7% of the cell lines tested and the lowest among all HCC cell lines tested (Table 1), we chose first to evaluate the antitumor effect of Triptonide in a HepG2-based orthotopic xenograft tumor model for human HCCs to provide an assessment for HCCs and hopefully for the majority cases of the 10 types of cancers.

The essence of targeted anticancer therapy is the selectively enhanced killing of cancer cells for a desirable therapeutic gain with minimal or no adverse effects. Thus, we first determined the lethal dose and the maximum tolerable dose (MTD) of Triptonide for mice based on a once every other day regimen. We found that 200 mg/kg resulted in 100% death within four days, while 100 mg/kg did not result in any significant adverse effects on the mice, establishing the lethal dose and the MTD within the 100 to 200 mg/kg range for this specific one every other day regimen. Meanwhile, we also found that when Triptonide was given at 100 mg/kg but for three times a day with a four-hour interval between each treatment, all animals were dead within four days. Together, these data showed that excessive exposure either at too high a dose or at a too short interval could increase the risk of general toxicity, while repeated dosing of up to 100 mg/kg at the appropriate intervals such as once every other day were well tolerated. It is noted that once every other day might not be the optimal scheme. Nonetheless, we decided to begin with this scheme and the 25% value of the MTD, i.e. 25 mg/kg.

A preliminary experiment was carried out to test the potential effective antitumor range of dosing by treating the tumor-bearing mice with 0, 1, 5, 10, 25 mg/kg by gavage with the regimen of one dosage every other day. The results of this preliminary experiment showed that compared to the 0 mg/kg control, treatments with 1 or 5 mg/kg had no significant effects on the changes in the intensities of the tumor specific signal; and those with 10 mg/kg reduced the tumor specific signals. In contrast, the treatments with 25 mg/kg Triptonide could not only rapidly reduce the intensity of the tumor specific signals, but eventually reduced the tumor specific signal to the background level (data not shown). Based on the result from this preliminary experiment, we concluded that the treatment with a single gavage dose of 25 mg/kg once every other day was sufficient to provide a potent anti-tumor effect.

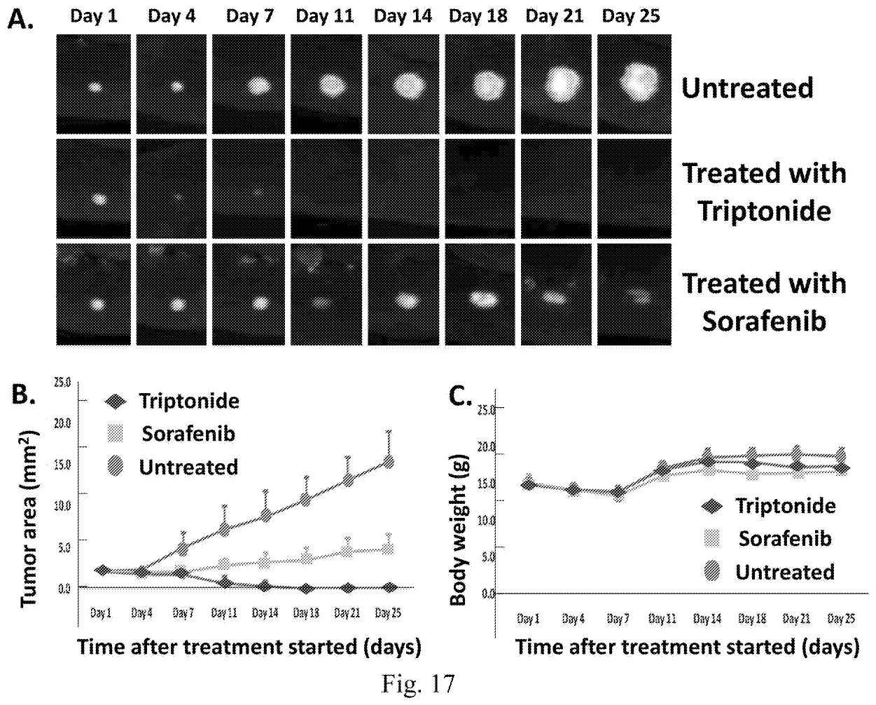

A full fledge preclinical experiment with a Green fluorescent protein (GFP)-expressing HepG2 (HepG2-GFP) orthotopic xenograft tumor model was then carried out. In this case, GFP was used as a tumor specific signal to assess the relative volumes of individual tumors. Thirty mice were randomly divided into three cohorts, 10 mice per cohort; and were treated with the vehicle only, Triptonide at the level of 25 mg/kg body weight, or Sorafenib, respectively. Sorafenib is the only anticancer drug approved by the USFDA for HCCs. It was therefore included as a positive control. The result showed that compared with the vehicle control group, the Sorafenib-treated group exhibited lower intensities of the tumor specific signals at all time points, indicative of a tumor suppressive effect. However, the tumor specific signals remained detectable and exhibited a trend of increased intensities over time for all treated mice throughout the treatment period. In contrast, the Triptonide-treated group was characterized by an initial rapid reduction in the intensities of the tumor specific signals. Remarkably, the tumor specific signals could no longer be detected in any of the ten mice by two weeks after the initiation of the treatment (FIG. 17A-17B). The representative imaging data for a single animal for each of the three cohorts are shown in FIG. 9A. Also, the treatment with Triptonide did not have any significant impact on the average body weight of the animals (FIG. 17C), indicating the lack of any major adverse effects. Autopsy of six of these mice did not reveal any signs of tumors, confirming the complete or near complete elimination of the tumor masses from these mice. Moreover, the remaining mice were monitored for another four months without further treatments. No tumor specific signals were ever detected again during this period.

Together, these data have indicated that the treatment with Triptonide effectively eliminated the tumors without causing any adverse effects to the tumor-baring animals.

### Discussion and Conclusions

In an attempt to identify candidates to be used for targeted anticancer therapy for human HCCs, we discovered that Triptonide had the desirable cancer cell-specific growth inhibitory effect (FIG. 1). The follow-up studies revealed that this specific growth inhibitory effect is due to Triptonide's unique mitotic catastrophe-inducing effect on mitogenically activated cells while sparing the quiescent, non-dividing cells (FIGS. 2-4).

We next showed that this unique mitotic catastrophe-inducing effect of Tritonide was distinct from the cell killing effect of Triptolide (FIG. 4 and FIG. 5), a well-studied anti-leukemia/anti-cancer agent, which causes primarily the cell cycle arrest of G1 cells and the apoptosis of S phase cells (FIG. 6). In cultured mouse keratinocytes, this unique effect of Triptonide was dependent on Par2 (FIGS. 7-8), a GPCR receptor. At the nanomoles range with one-hour exposure, Triptonide, but not Triptolide exhibited this unique Par2-dependent effect (FIG. 9). Together, these data define Triptonide as a unique agent that can be used to induce mitotic catastrophe in Par2/PAR2-expressing cells, leading to the selective killing of proliferating Par2/PAR2-expressing cells.

Mechanistically, Triptonide could cause the activation of ERK (FIG. 10), which promotes G1/S transition, as well as the inhibition of CDK1 activation prior to mitotic entry (G2/M transition) (FIG. 11). Thus, Triptonide could first promote G1/S transition and then block G2/M transition. The blocking effect of Triptonide on G2/M transition is responsible for its unique mitotic catastrophe-inducing effect. Specifically, Triptonide caused the sustained activation of the AC-cAMP-PKA pathway and sustained high activity of PKA prior to G2/M transition (FIG. 12-14). Inhibition of AC-cAMP-PKA pathway protected HepG2 cells from the mitotic catastrophe-inducing effect of Triptonide (FIG. 15). Accordingly, given that growth inhibitory effect of Triptonide (caused by mitotic catastrophe) is also dependent on Par2 (FIG. 7), it then became evident that the mitotic catastrophe-inducing effect of Triptonide is due to its agonist effect on the Par2/PAR2-Gαs-AC-cAMP-PKA signaling cascade.

Taken together, these data have led to the discovery of a new paradigm for instigating the selective killing of proliferating PAR2-expressing by targeting PAR2 with Triptonide and/or other PAR2 agonist(s) that could be used to cause the unusual activation of the AC-cAMP-PAK signaling pathway, while sparing PAR2 negative cells as well as those PAR2 expressing cells that are not proliferating. In addition, since individual cells are only vulnerable to this killing effect of Triptonide, the ability to activate ERK could increase the efficacy of the killing by promoting the entry of quiescent cells into the cell cycle.

The inventors next found that at least one cell line from a collection of human cancer cell lines that represent the 12 major types of human cancers was more sensitive than HepG2 (Table 1). Moreover, PAR2 expression was detected in all gastric cancer cell lines examined (FIG. 16). Together, these data reveal that PAR2 expression is a common feature of at least a subset of all these major types of human cancers. Accordingly, the new paradigm for instigating the selective killing of cancer cells is likely to be applicable to all these cancer types. Thus, the Triptonide-PAR2 paradigm could be applicable for instigating the killing of cancer cells for many, or perhaps all types of human cancers.

Intriguingly, the inventors also revealed that PAR2 is expressed in both of the two immortalized cell line examined, suggesting that acquisition of PAR2 expression is an early event for many cancers (FIG. 8 and FIG. 16). In another words, PAR2 is a shared feature in all cells of a PAR2-expressing tumor and is a good target for developing targeted anticancer treatments.

Significantly, the results from a preclinical experiment showed that a Triptonide single agent therapy had a curative effect on an orthotopical xenograft liver tumor model. Remarkably, the curative effect was achieved with a drug dose that is four folds lower than the maximum non-lethal dose. Together, these data demonstrate that Triptonide can be used to instigate the selective and effective killing of cancer cells for therapeutic gain with little or no adverse side effects. It should be noted that HepG2 has a median IC50 for Triptonide (IC50: 7.509 μM) that is higher than 68% of the cancer cell lines examined (Table 1), suggesting that Triptonide, or its functional equivalents, could potentially be used to treat many types of cancers.

Intriguingly, the one-hour exposure of 1 μM Triptonide to HepG2 cells around the S/G2 (or perhaps also Gl/S) transition caused mitotic catastrophe at a time frame that was coincided with the timing for mitosis. Significantly, this unique mechanism of cell death restricts its damage to only the cycling portion of the PAR2-expressing cells. As PAR2 is primarily expressed in non-cycling postmitotic cells in humans, this has raised the possibility that Triptonide, or other agents with the same unique effect on PAR2, could be used to implement the specific killing of cycling PAR2-expressing cancer cells without inflicting any significant adverse effects to any of the non-cancer cells including the non-cycling PAR2-expressing cells. Thus, taken together, our data have established that the non-canonical activation of PAR2, in a manner that is mediated by Triptonide, constitutes a new candidate paradigm for developing targeted anticancer therapy.

The concept of targeting PAR2 with specific, biased or abnormal agonists to achieve cancer-cell-specific killing for targeted anticancer therapy has not been reported to date. It also represents the first example for the effectiveness of targeting a GPCR for anticancer benefit. And to the best of our knowledge, the instant application also constitutes the first disclosure for the identification of small molecular agonists for the PAR2-AC-cAMP-PKA signaling pathway.

The finding that Triptonide has such a unique anticancer property was a major surprise for us. Remarkably, our data clearly showed that Triptolide and Triptonide exhibited contrasting different effects both on ERK phosphorylation in HepG2 cells and on the growth and survival of Par2 knockout keratinocytes. Accordingly, while the antitumor activity of Triptolide that is due to its general, non-cancer cell-specific cytotoxicity, has been well known, the unique mode of PAR2-dependent cytotoxicity and the targeted antitumor activity of Triptonide described here have not been reported previously.

Furthermore, the use of the cultured primary mouse hepatocytes rather than an immortalized cell line, such as LO2, has proven critical in this discovery, since PAR2 is apparently expressed in the immortalized LO2 cell line (interestingly also the immortalized cell line GES-1). Of note, when applied in the usual continuous exposure fashion, Triptonide did not exhibit any significant cancer cell-enhanced killing effect. Serendipitously, by employing a unique screening strategy that combines the use of primary cultured mouse hepatocytes as a non-cancer control and the one-hour drug treatment scheme, we were very fortunate to have discovered the differential effects of Triptonide on primary cultured mouse hepatocytes and its presumed cancer counterpart HepG2 cells in our initial experiment.

The subsequent finding that a Triptonide-based treatment at a dose that was four fold below the maximal tolerable dose resulted in a full curative outcome was indeed a surprise given the potential off-target cytotoxic effect(s) of the drug. A drug that targets a cell surface receptor could exhibit such a target-only detrimental effect if it is highly specific for the desirable target of interest; and/or if it has a pharmacokinetic attribute that would limit its access to this desirable target. In this case, it is worth noting that in rodents, Triptonide, when administered via intraperitoneal injection, has a very short T1/2 alpha (0.17-0.195 hours) and T1/2 beta (about 4.95-6.49 hours). It is also worth noting that there has been great interest in the anti-cancer activity of Triptolide, despite its inherent general cytotoxicity. However, it has become increasingly evident that the very narrow range between the effective doses of anti-tumor activities and the minimal dose of general toxicity could represent a major hurdle for its clinical application as an anticancer drug. In contrast, Triptonide is not only less potent in terms of its general cytotoxicity (i.e. PAR2-independent toxicity), but also it has very unique pharmacokinetic characteristics such as rapid redistribution/metabolism and fast clearance. These unique features might prove to be yet another desirable attribute as a drug for targeting PAR2 (a cell surface receptor), for the selective killing of PAR2-expressing cancer cells through targeted anticancer therapies.

It is likely that other compounds or biologic agents could be used to cause similar PAR2-dependent killing of proliferating cells. Interestingly, a previous study has implicated Triptolide as a potent inhibitor for PAR2-dependent functions, while our data indicate that Triptonide causes the PAR2-dependent killing by acting as an abnormal activator for PAR2.

In retrospect, given the widespread expression of Par2 in several cell types across many types of organs/tissues, the finding that a treatment with a PAR2 agonist for the Gαs-AC-cAMP-PKA would be expected to cause the elevation of PKA in all these PAR2-expresing cells. Accordingly, our data have also led to the realization that the up-regulation of the PKA activity in these mouse cells is apparently well tolerated. It is likely that such a feature could be common to many other cells types and could be conserved between mouse and humans. In this regards, it is reasonable to predict that other GPCR receptors, which are expressed in human cancer cells, could be exploited for instigating the effective killing of the cancer cells for therapeutic gains with manageable or no major adverse side effects. Likewise, other components that can be used to up-regulate PKA activities in cancer cells, in a manner similar to that of Triptonide, could potentially be exploited for anticancer benefits.

In addition, it is highly likely that Triptonide and/or its functional analogues could be combined with other drugs or modalities including immunotherapies to develop new anti-cancer treatment strategies.

In summary, our studies supported that the sustained elevation of PKA activities through the activation of PAR2 GPCR receptor by Triptonide could be used as a novel strategy for targeted anticancer therapy. We have succeeded in the development and implementation of this strategy, providing the proof of concept for developing personalized anticancer therapies through the use of Triptonide and possibly also other compounds or biologics that have a similar effect on PAR2, either by themselves or in the setting of combinatorial therapies with other agents and/or modalities.

As the sustained elevation of PKA activity could potentially be achieved in many different ways, elevation of PKA could be exploited as a general anticancer paradigm. In particular, many GPCR receptors are coupled to the Gαs-AC-cAMP-PKA signaling cascade and some are expressed in some human cancers. Although under normal physiological conditions, the activation of individual GPCR receptors by their cognate ligands usually results in the up-regulation of PKA activity in a highly controlled manner, it remains possible that some biased ligands for such GPCR receptors could cause a sustained activation of PKA activity just as the activation of PAR2 by Triptonide does. Accordingly, such biased GPCR ligands could be used to instigate the selective killing of cancer cells for therapeutic gain as well. In this regard, the methods described herein for the detection of the effect of Triptonide at the cell biology level could prove useful in identifying new agents, including functional analogs of Triptonide, that can be used to cause the mitotic catastrophe-inducing effect. Such a functional assay as well as the related methods will have important applications in developing companion diagnostic tests that can be used to guide patient selection in the clinical settings.

It is understood that, although the inventions have been described in some detail by way of illustration and examples, these inventions are not limited to specific details described in these specific forms. Apparently to those skilled in this field, various equivalent changes and modifications to the technical features involved in the inventions may be practiced without deviating from the spirit of the inventions described herein, and such changes and modifications are within the scope of the inventions.

### REFERENCES

1. Hahn, A. T., Jones, J. T. and Meyer, T. (2009) Quantitative analysis of cell cycle phase durations and PC12 differentiation using fluorescent biosensors. Cell Cycle, 8, 1044-1052.

2. Larsson, O. and Zetterberg, A. (1995) Existence of a commitment program for mitosis in early G1 in tumour cells. Cell Prolif, 28, 33-43.

3. Martinsson, H. S., Starborg, M., Erlandsson, F. and Zetterberg, A. (2005) Single cell analysis of G1 check points-the relationship between the restriction point and phosphorylation of pRb. Exp Cell Res, 305, 383-391.

4. Schwarz, C., Johnson, A., Koivomagi, M., Zatulovskiy, E., Kravitz, C. J., Doncic, A. and Skotheim, J. M. (2018) A Precise Cdk Activity Threshold Determines Passage through the Restriction Point. Mol Cell, 69, 253-264 e255.

5. Yao, G., Lee, T. J., Mori, S., Nevins, J. R. and You, L. (2008) A bistable Rb-E2F switch underlies the restriction point. Nat Cell Biol, 10, 476-482.

6. Matson, J. P. and Cook, J. G. (2017) Cell cycle proliferation decisions: the impact of single cell analyses. FEBS J, 284, 362-375.

7. Malumbres, M. and Barbacid, M. (2005) Mammalian cyclin-dependent kinases. Trends Biochem Sci, 30, 630-641.

8. Potapova, T. A., Sivakumar, S., Flynn, J. N., Li, R. and Gorbsky, G. J. (2011) Mitotic progression becomes irreversible in prometaphase and collapses when Wee1 and Cdc25 are inhibited. Mol Biol Cell, 22, 1191-1206.

9. Chin, C. F. and Yeong, F. M. (2010) Safeguarding entry into mitosis: the antephase checkpoint. Mol Cell Biol, 30, 22-32.

10. Andreassen, P. R., Lohez, O. D. and Margolis, R. L. (2003) G2 and spindle assembly checkpoint adaptation, and tetraploidy arrest: implications for intrinsic and chemically induced genomic instability. Mutat Res, 532, 245-253.

11. Gavet, O. and Pines, J. (2010) Activation of cyclin B1-Cdkl synchronizes events in the nucleus and the cytoplasm at mitosis. J Cell Biol, 189, 247-259.

12. Gavet, O. and Pines, J. (2010) Progressive activation of CyclinB 1-Cdk1 coordinates entry to mitosis. Dev Cell, 18, 533-543.

13. Han, S. J. and Conti, M. (2006) New pathways from PKA to the Cdc2/cyclin B complex in oocytes: Wee1B as a potential PKA substrate. Cell Cycle, 5, 227-231.

14. Solc, P., Schultz, R. M. and Motlik, J. (2010) Prophase I arrest and progression to metaphase I in mouse oocytes: comparison of resumption of meiosis and recovery from G2-arrest in somatic cells. Mol Hum Reprod, 16, 654-664.

15. Wen, W., Taylor, S. S. and Meinkoth, J. L. (1995) The expression and intracellular distribution of the heat-stable protein kinase inhibitor is cell cycle regulated. J Biol Chem, 270, 2041-2046.

16. Malumbres, M. and Barbacid, M. (2009) Cell cycle, CDKs and cancer: a changing paradigm. Nat Rev Cancer, 9, 153-166.

17. Mills, G. B. (2012) An emerging toolkit for targeted cancer therapies. Genome Res, 22, 177-182.

18. Knepper, T. C., Saller, J. and Walko, C. M. (2017) Novel and Expanded Oncology Drug Approvals of 2016-PART 1: New Options in Solid Tumor Management. Oncology (Williston Park), 31, 110-121.

19. Gross, S., Rahal, R., Stransky, N., Lengauer, C. and Hoeflich, K. P. (2015) Targeting cancer with kinase inhibitors. J Clin Invest, 125, 1780-1789.

20. Druker, B. J. (2008) Translation of the Philadelphia chromosome into therapy for CML. Blood, 112, 4808-4817.

21. Liegl-Atzwanger, B., Fletcher, J. A. and Fletcher, C. D. (2010) Gastrointestinal stromal tumors. Virchows Arch, 456, 111-127.

22. McDermott, U. and Settleman, J. (2009) Personalized cancer therapy with selective kinase inhibitors: an emerging paradigm in medical oncology. J Clin Oncol, 27, 5650-5659.

23. Gerber, D. E. and Minna, J. D. (2010) ALK inhibition for non-small cell lung cancer: from discovery to therapy in record time. Cancer Cell, 18, 548-551.

24. Tannock, I. F. and Hickman, J. A. (2016) Limits to Personalized Cancer Medicine. N Engl J Med, 375, 1289-1294.

25. Swanton, C., Soria, J. C., Bardelli, A., Biankin, A., Caldas, C., Chandarlapaty, S., de Koning, L., Dive, C., Feunteun, J., Leung, S. Y. et al. (2016) Consensus on precision medicine for metastatic cancers: a report from the MAP conference. Ann Oncol, 27, 1443-1448.

26. Muller, G. (2000) Towards 3D structures of G protein-coupled receptors: a multidisciplinary approach. Curr Med Chem, 7, 861-888.

27. Hopkins, A. L. and Groom, C. R. (2002) The druggable genome. Nat Rev Drug Discov, 1, 727-730.

28. Coughlin, S. R. (2000) Thrombin signalling and protease-activated receptors. Nature, 407, 258-264.

29. Soh, U. J., Dores, M. R., Chen, B. and Trejo, J. (2010) Signal transduction by protease-activated receptors. Br J Pharmacol, 160, 191-203.

30. Rothmeier, A. S. and Ruf, W. (2012) Protease-activated receptor 2 signaling in inflammation. Semin Immunopathol, 34, 133-149.

31. Ramachandran, R., Altier, C., Oikonomopoulou, K. and Hollenberg, M. D. (2016) Proteinases, Their Extracellular Targets, and Inflammatory Signaling. Pharmacol Rev, 68, 1110-1142.

32. Zhao, P., Metcalf, M. and Bunnett, N. W. (2014) Biased signaling of protease-activated receptors. Front Endocrinol (Lausanne), 5, 67.

33. Torres-Quesada, O., Mayrhofer, J. E. and Stefan, E. (2017) The many faces of compartmentalized PKA signalosomes. Cell Signal, 37, 1-11.

34. Taylor, S. S., Kim, C., Vigil, D., Haste, N. M., Yang, J., Wu, J. and Anand, G. S. (2005) Dynamics of signaling by PKA. Biochim Biophys Acta, 1754, 25-37.

35. Smith, J. S., Lefkowitz, R. J. and Rajagopal, S. (2018) Biased signalling: from simple switches to allosteric microprocessors. Nat Rev Drug Discov, 17, 243-260.

36. Geppetti, P., Veldhuis, N. A., Lieu, T. and Bunnett, N. W. (2015) G Protein-Coupled Receptors: Dynamic Machines for Signaling Pain and Itch. Neuron, 88, 635-649.

37. Bohm, S. K., Kong, W., Bromme, D., Smeekens, S. P., Anderson, D. C., Connolly, A., Kahn, M., Nelken, N. A., Coughlin, S. R., Payan, D. G. et al. (1996) Molecular cloning, expression and potential functions of the human proteinase-activated receptor-2. Biochem J, 314 (Pt 3), 1009-1016.

38. D'Andrea, M. R., Derian, C. K., Leturcq, D., Baker, S. M., Brunmark, A., Ling, P., Darrow, A. L., Santulli, R. J., Brass, L. F. and Andrade-Gordon, P. (1998) Characterization of protease-activated receptor-2 immunoreactivity in normal human tissues. J Histochem Cytochem, 46, 157-164.

39. Yau, M. K., Lim, J., Liu, L. and Fairlie, D. P. (2016) Protease activated receptor 2 (PAR2) modulators: a patent review (2010-2015). Expert Opin Ther Pat, 26, 471-483.

40. D'Andrea, M. R., Derian, C. K., Santulli, R. J. and Andrade-Gordon, P. (2001) Differential expression of protease-activated receptors-1 and -2 in stromal fibroblasts of normal, benign, and malignant human tissues. Am J Pathol, 158, 2031-2041.

41. Fujimoto, D., Hirono, Y., Goi, T., Katayama, K., Hirose, K. and Yamaguchi, A. (2006) Expression of protease activated receptor-2 (PAR-2) in gastric cancer. J Surg Oncol, 93, 139-144.

42. Su, S., Li, Y., Luo, Y., Sheng, Y., Su, Y., Padia, R. N., Pan, Z. K., Dong, Z. and Huang, S. (2009) Proteinase-activated receptor 2 expression in breast cancer and its role in breast cancer cell migration. Oncogene, 28, 3047-3057.

43. Regard, J. B., Sato, I. T. and Coughlin, S. R. (2008) Anatomical profiling of G protein-coupled receptor expression. Cell, 135, 561-571.

44. Wojtukiewicz, M. Z., Hempel, D., Sierko, E., Tucker, S. C. and Honn, K. V. (2015) Protease-activated receptors (PARs)—biology and role in cancer invasion and metastasis. Cancer Metastasis Rev, 34, 775-796.

45. DeFea, K. A., Zalevsky, J., Thoma, M. S., Dery, O., Mullins, R. D. and Bunnett, N. W. (2000) beta-arrestin-dependent endocytosis of proteinase-activated receptor 2 is required for intracellular targeting of activated ERK1/2. J Cell Biol, 148, 1267-1281.

46. Ruf, W., Rothmeier, A. S. and Graf, C. (2016) Targeting clotting proteins in cancer therapy—progress and challenges. Thromb Res, 140 Suppl 1, S1-7.

47. Kupchan, S. M., Court, W. A., Dailey, R. G., Jr., Gilmore, C. J. and Bryan, R. F. (1972) Triptolide and tripdiolide, novel antileukemic diterpenoid triepoxides from Tripterygium wilfordii. J Am Chem Soc, 94, 7194-7195.

48. Kupchan, S. M. and Schubert, R. M. (1974) Selective alkylation: a biomimetic reaction of the antileukemic triptolides? Science, 185, 791-793.

49. Kupchan, S. M. (1976) Novel plant-derived tumor inhibitors and their mechanisms of action. Cancer Treat Rep, 60, 1115-1126.

50. Ma, J., Dey, M., Yang, H., Poulev, A., Pouleva, R., Dorn, R., Lipsky, P. E., Kennelly, E. J. and Raskin, I. (2007) Anti-inflammatory and immunosuppressive compounds from Tripterygium wilfordii. Phytochemistry, 68, 1172-1178.

51. Shamon, L. A., Pezzuto, J. M., Graves, J. M., Mehta, R. R., Wangcharoentrakul, S., Sangsuwan, R., Chaichana, S., Tuchinda, P., Cleason, P. and Reutrakul, V. (1997) Evaluation of the mutagenic, cytotoxic, and antitumor potential of triptolide, a highly oxygenated diterpene isolated from Tripterygium wilfordii. Cancer Lett, 112, 113-117.

52. Zheng, J. (1991) [Screening of active anti-inflammatory, immunosuppressive and antifertility components from Tripterygium wilfordii. IV. A comparison of the male antifertility activities of 7 diterpene lactone epoxide compounds]. Zhongguo Yi Xue Ke Xue Yuan Xue Bao, 13, 398-403.

53. Liu, Z., Ma, L. and Zhou, G. B. (2011) The main anticancer bullets of the Chinese medicinal herb, thunder god vine. Molecules, 16, 5283-5297.

54. Leuenroth, S. J. and Crews, C. M. (2005) Studies on calcium dependence reveal multiple modes of action for triptolide. Chem Biol, 12, 1259-1268.

55. Hyun, E., Andrade-Gordon, P., Steinhoff, M., Beck, P. L. and Vergnolle, N. (2010) Contribution of bone marrow-derived cells to the pro-inflammatory effects of protease-activated receptor-2 in colitis. Inflamm Res, 59, 699-709.

56. Banfalvi, G. (2017) Overview of Cell Synchronization. Methods Mol Biol, 1524, 3-27.

57. Stalheim, L., Ding, Y., Gullapalli, A., Paing, M. M., Wolfe, B. L., Morris, D. R. and Trejo, J. (2005) Multiple independent functions of arrestins in the regulation of protease-activated receptor-2 signaling and trafficking. Mol Pharmacol, 67, 78-87.

58. Glass, D. B., Cheng, H. C., Mende-Mueller, L., Reed, J. and Walsh, D. A. (1989) Primary structural determinants essential for potent inhibition of cAMP-dependent protein kinase by inhibitory peptides corresponding to the active portion of the heat-stable inhibitor protein. J Biol Chem, 264, 8802-8810.

59. Gang, Y. Y., Zhang, Z. X., Zhang, S. Q., Liu, X. D. and An, D. K. (1996) [Pharmacokinetics and disposition of triptonide in rats]. Yao Xue Xue Bao, 31, 901-905.

60. Kupchan, S. M., Sigel, C. W., Matz, M. J., Gilmore, C. J. and Bryan, R. F. (1976) Structure and stereochemistry of jatrophone, a novel macrocyclic diterpenoid tumor inhibitor. J Am Chem Soc, 98, 2295-2300.

61. Bailey, L. J., Choudhary, V., Merai, P. and Bollag, W. B. (2014) Preparation of primary cultures of mouse epidermal keratinocytes and the measurement of phospholipase D activity. Methods Mol Biol, 1195, 111-131.

62. Luo, Z., Yu, G., Lee, H. W., Li, L., Wang, L., Yang, D., Pan, Y., Ding, C., Qian, J., Wu, L. et al. (2012) The Nedd8-activating enzyme inhibitor MLN4924 induces autophagy and apoptosis to suppress liver cancer cell growth. Cancer Res, 72, 3360-3371.

1. A method for treating or preventing hyperproliferative disorders, immune response related disorders and/or for pain control in a subject, comprising administering to the subject a therapeutically or prophylactically effective amount of an agent that can cause activation of protein kinase A.

2. The method according to claim 1, wherein the treatment selectively kills cancer cells.

3. A pharmaceutical composition comprising Triptonide or a functional equivalent or pharmaceutically acceptable salt thereof, and a pharmaceutically acceptable carrier.

4. (canceled)

5. (canceled)

6. The pharmaceutical composition according to claim 3, wherein the pharmaceutical composition is formulated into a pharmaceutically acceptable dosage form.

7. (canceled)

8. (canceled)

9. The method according to claim 2, wherein the cancer is selected from hepatocellular carcinoma, breast cancer, colon cancer, non-small cell lung cancer, gastric cancer, ovarian cancer, renal cancer, prostate cancer, central nerve system cancer and melanoma.

10. (canceled)

11. (canceled)

12. (canceled)

13. (canceled)

14. (canceled)

15. A method for inducing a sustained activation of PKA in PAR2-expressing proliferating cells, comprising contacting the cells with Triptonide, or a functional equivalent or pharmaceutically acceptable salt thereof, or a pharmaceutical composition comprising the same.

16. The method according to claim 1, wherein the agent is Triptonide, or a functional equivalent or pharmaceutically acceptable salt thereof, or a pharmaceutical composition comprising Triptonide, or a functional equivalent or pharmaceutically acceptable salt thereof.

17. The method according to claim 1, wherein the hyperproliferative disorders are cancers.

18. The method according to claim 1, wherein the prevention comprises selectively killing PAR2-expressing cells in premalignant and/or malignant sites.

19. The method according to claim 2, wherein the cancer cells are protease activated receptor 2 (PAR2)-expressing proliferating cells.

20. The pharmaceutical composition according to claim 3, wherein the composition further comprises one or more additional agents that cause activation of protein kinase A.

21. The pharmaceutical composition according to claim 6, wherein the pharmaceutically acceptable dosage form is selected from oral liquids, capsules, powders, tablets, granules, pills, syrups and injections.

22. The method according to claim 16, wherein the Triptonide, or a functional equivalent or pharmaceutically acceptable salt thereof, or a pharmaceutical composition comprising the same is administered orally, subcutaneously, intramuscularly or intraperitoneally.

23. The method according to claim 17, wherein the cancers are selected from hepatocellular carcinoma, breast cancer, colon cancer, non-small cell lung cancer, gastric cancer, ovarian cancer, renal cancer, prostate cancer, central nerve system cancer and melanoma.
